(19) United States
(12) Patent Application Publication
(10) Pub. No.: US 2023/0167406 A1
(43) Pub. Date: Jun. 1, 2023

(54) COMPOSITIONS AND METHODS OF PROMOTING MYELINATION

(71) Applicant: The Children's Medical Center Corporation, Boston, MA (US)

(72) Inventors: Zhigang HE, Boston, MA (US); Jing WANG, Boston, MA (US); Xuelian HE, Boston, MA (US)

(73) Assignee: The Children's Medical Center Corporation, Boston, MA (US)

(21) Appl. No.: 17/921,802

(22) PCT Filed: Apr. 29, 2021
(86) PCT No.: PCT/US2021/029806

§ 371 Date: Oct. 27, 2022

Related U.S. Application Data
(60) Provisional application No. 63/018,939, filed on May 1, 2020.

(51) Int. Cl. C12N 5/079; A61P 25/00

(52) U.S. Cl. CPC C12N 5/0622; A61P 25/00; C12N 2501/999

(57) ABSTRACT

The invention features methods and compositions that are useful for the treatment of diseases, disorders, conditions, or injuries characterized by insufficient myelination. The methods involve administering GPR17 antagonists and microglia inhibitors or ablation agents.

### CROSS REFERENCE TO RELATED APPLICATIONS

This application is an International PCT Application, which claims priority to and the benefit of U.S. Provisional Application Ser. No. 63/018,939 filed May 1, 2020, the entire contents of which are hereby incorporated by reference in its entirety.

### STATEMENT OF RIGHTS TO INVENTIONS MADE UNDER FEDERALLY SPONSORED RESEARCH

This invention was made with government support under Grant No. 5R01EY026939 awarded by the National Institutes of Health. The government has certain rights in the invention.

### BACKGROUND OF THE INVENTION

Functional deficits associated with CNS injuries, such as traumatic brain injury and spinal cord injury, have been largely attributed to the severing of long-projection axons and subsequent the disruptions of relevant circuits. However, despite tremendous progress in developing strategies of promoting axon regeneration, the achieved behavioral improvements with these methods are still limited even in experimental injury models. For example, regenerating axons induced by manipulations of enhancing the intrinsic regenerative ability of retinal ganglion cells (RGCs) are able to make functional synapses in their appropriate targets such as superior colliculus, but remain unmyelinated. In light of well-established role of myelin in facilitating axon conduction, these observations suggest the myelination failure of regenerated axons as an under-appreciated barrier of functional recovery and point to a need to understand the regulatory mechanisms of myelination of regenerated axons in the adult.

It is well known that myelination does not cease after the completion of neural development but continue to occur in the adult CNS. This is achieved by oligodendrocyte precursor cells (OPCs), which are ubiquitously distributed in all parts of CNS. For successful myelination, residential OPCs often undergo proliferation, followed by a multi-step yet poorly understood differentiation process, ultimately becoming myelination-competent oligodendrocytes. The onset and timing of CNS differentiation and myelination is tightly regulated by both intrinsic and extrinsic factors. On the other hand, myelination failure underlies a number of neurological diseases, such as multiple sclerosis (MS), leukodystrophies, and neurodegenerative Alzheimer's disease. For example, in the advanced stage of MS, called progressive MS, some proliferating OPCs remain in the lesion core but fail to differentiate into mature oligodendrocytes. Therefore, numerous efforts have been devoted to developing strategies of promoting the proliferation and differentiation of OPCs. However, in most commonly used demyelination models such as experimental allergic encephalomyelitis (EAE), certain degrees of remyelination could occur spontaneously, leaving it challenging to tell if any pro-myelination treatment acts by speeding up the spontaneous process and/or initiating de novo myelination. Furthermore, the nature of multi-step differentiation required for an OPC to mature oligodendrocytes may demand the manipulations dealing with more than one steps.

Currently, there is no effective treatment for diseases associated with myelination failure, such as multiple sclerosis (MS), leukodystrophies, and neurodegenerative Alzheimer's disease, or for central nervous system injuries (e.g., traumatic brain injury, spinal cord injury) associated with myelination failure.

### SUMMARY OF THE INVENTION

As described below, the present invention features methods and compositions that are useful for treating diseases, disorders, conditions, and injuries characterized by insufficient myelination.

In one aspect, the invention provides a method for increasing myelination of an axon, the method involving contacting an oligodendrocyte progenitor cell (OPC) in the presence of an axon with an agent that inhibits GPR17 and/or an agent that ablates and/or inhibits an activated microglia, thereby increasing myelination of the axon. In some embodiments, the axon is injured and/or demyelinated.

In another aspect, the invention provides a method of increasing myelination of an axon, the method involving contacting an oligodendrocyte progenitor cell (OPC) in the presence of an axon with an agent that inhibits GPR17 and/or an agent that inhibits TNFα Receptor 2 or TNFα, thereby increasing myelination of the axon. In some embodiments, the agent that inhibits TNFα Receptor 2 or TNFα is thalidomide.

In yet another aspect, the invention provides a method for increasing OPC number and/or differentiation, the method involving contacting an oligodendrocyte progenitor cell (OPC) with an agent that inhibits GPR17 and/or an agent that ablates or inhibits an activated microglia, thereby increasing OPC number and/or differentiation. In some embodiments of any of the above aspects, the agent that inhibits GPR17 is Montelukast or Pranlukast.

In still another aspect, the invention provides a method for increasing OPC number and/or differentiation, the method involving contacting an oligodendrocyte progenitor cell (OPC) with an agent that inhibits GPR17 and/or an agent that inhibits TNFα Receptor 2 or TNFα, thereby increasing OPC number and/or differentiation. In some embodiments of any of the above aspects, the agent that ablates or inhibits an activated microglia is PLX3397. In some embodiments, the agent that inhibits TNFα Receptor 2 or TNFα is thalidomide.

In another aspect the invention provides a method of increasing myelination of an axon, the method involving contacting a oligodendrocyte progenitor cell (OPC) in the presence of an axon with an agent that is any one or more of benztropine mesylate, clemastine, Montelukast, Pranlukast, and thalidomide, thereby increasing myelination of the axon. In some embodiments, the agent that inhibits GPR17 is montelukast.

In another aspect the invention provides a method of increasing OPC number and/or differentiation, the method involving contacting a oligodendrocyte progenitor cell (OPC) with an agent that is any one or more of benztropine mesylate, clemastine, Montelukast, Pranlukast, and thalidomide, thereby increasing OPC number and/or differentiation. In some embodiments, the agent that inhibits GPR17 is montelukast.

In some embodiments of any of the above aspects, the method increases the number of CC1 and/or Oligo1 positive OPCs. In some embodiments, the agents are administered concurrently or sequentially. In some embodiments, the agent that inhibits GPR17 is administered simultaneously with the agent that ablates or inhibits an activated microglia. In some embodiments, the agent that inhibits GPR17 is administered at least one week prior to the agent that ablates or inhibits the activated microglia. In some embodiments, the agents are administered prior to, concurrent with, or subsequent to injury. In some embodiments, the agents are administered days or weeks subsequent to injury. In some embodiments, the agents are administered 1-2 weeks subsequent to injury. In some embodiments, the agents are administered for at least between 14 and 28 days. In some embodiments, the method is performed in vivo or in vitro.

In another aspect, the invention provides a method of increasing myelination of an axon in a subject, the method involving administering to the subject an agent that inhibits GPR17 and/or an agent that ablates or inhibits an activated microglia, thereby increasing myelination of the axon.

In another aspect the invention provides a method of increasing OPC number and/or differentiation in a subject, the method involving administering to the subject an agent that inhibits GPR17 and/or an agent or inhibits that ablates or inhibits an activated microglia, thereby increasing OPC number and/or differentiation. In some embodiments of the previous two aspects, the agent that inhibits GPR17 is Montelukast or Pranlukast. In some embodiments, the agent that ablates or inhibits an activated microglia is PLX3397.

In another aspect, the invention provides a method for increasing myelination of an axon in a subject in need thereof, the method involving administering to the subject an agent that is any one or more of benztropine mesylate, clemastine, Montelukast, Pranlukast, and thalidomide, thereby increasing myelination of the axon.

In another aspect, the invention provides a method of increasing OPC number and/or differentiation in a subject in need thereof, the method involving administering to the subject an agent that is any one or more of benztropine mesylate, clemastine, Montelukast, Pranlukast, and thalidomide, thereby increasing OPC number and/or differentiation.

In another aspect, the invention provides a method of treating a subject having a disease or injury associated with myelination failure, the method involving administering to the subject an agent that inhibits GPR17 and/or an agent that ablates or inhibits an activated microglia. In some embodiments of any of the above aspects or any other aspect of the invention herein, the method increases the number of CC1 and/or Oligo1 positive OPCs. In some embodiments of any of the above aspects or any other aspect of the invention herein, the subject has a disease associated with a failure in myelination, e.g., multiple sclerosis (MS), a leukodystrophy, neurodegenerative Alzheimer's disease, traumatic brain injury, spinal cord injury, or optic nerve injury. In some embodiments, the leukodystrophy is one Adrenoleukodystrophy (ALD), Aicardi-Goutieres Syndrome, Alexander Disease, Canavan Disease, Cerebrotendinous Xanthomatosis (CTX), Globoid Cell Leukodystrophy (Krabbe Disease), Metachromatic Leukodystrophy (MLD), Pelizaeus Merzbacher Disease (X-linked spastic paraplegia), or Childhood Ataxia with Central Nervous System Hypomyelination (CACH).

In some embodiments of any of the above aspects or any other aspect of the invention herein, the agents are administered concurrently or sequentially. In some embodiments, the agent that inhibits GPR17 is administered simultaneously with the agent that ablates or inhibits an activated microglia. In some embodiments, the agent that inhibits GPR17 is administered at least one week prior to the agent that ablates or inhibits the activated microglia. In some embodiments, the agents are administered prior to, concurrent with, or subsequent to injury. In some embodiments, the agents are administered days or weeks subsequent to injury. In some embodiments, the agents are administered 1-2 weeks subsequent to injury. In some embodiments, the traumatic brain injury is a concussion. In some embodiments, the oligodendrocyte precursor cell is CC1− and has Oligo1 localized in the nucleus. In some embodiments, the OPC is an early differentiated oligodendrocyte that is CC1+ and has Oligo1 localized in the nucleus.

In some embodiments of any of the preceding aspects, the OPC is a differentiated oligodendrocyte that is CC1+ and has Oligo1 localized in the cytoplasm.

In yet another aspect, the invention provides a composition having a GPR17 antagonist and a microglia inhibitor or ablation agent, a TNFα Receptor 2 inhibitor, or a TNFα inhibitor. In some embodiments, the GPR17 antagonist is montelukast. In some embodiments, the microglia inhibitor or ablation agent is PLX3397. In some embodiments, the TNFα inhibitor is thalidomide.

In yet another aspect, the invention provides a method of identifying a compound that elicits differentiation of an oligodendrocyte or an oligodendrocyte precursor cell, the method involving injuring the optic nerve of a mouse, contacting the optic nerve with an agent that regenerates an axon; administering a candidate compound to the mouse to elicit differentiation of an oligodendrocyte precursor cell; administering a known microglia inhibitor or ablation agent; and determining the differentiation status of oligodendrocytes or oligodendrocyte precursor cells, where an increase in CC1+ oligodendrocytes relative to an untreated control indicates that the candidate compound elicited differentiation of the oligodendrocyte precursor cell.

In yet another aspect, the invention provides a method is provided for identifying a compound that elicits differentiation of an oligodendrocyte or an oligodendrocyte precursor cell, the method involving injuring the optic nerve of a mouse, contacting the optic nerve with an agent that regenerates an axon, administering a compound to the mouse that is known elicit differentiation of an oligodendrocyte precursor cell, administering a suspected microglia inhibitor or ablation agent, and determining the differentiation status of oligodendrocytes or oligodendrocyte precursor cells, where an increase in CC1+ oligodendrocytes having cytoplasmic Oligo1 relative to an untreated control indicates that the suspected microglia inhibitor or ablation agent effectively inhibited or ablated microglia cells.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In one embodiment, the agent is a GPR17 antagonist or a microglia inhibitor or ablation agent. In another embodiment, the agent is a compound that increases (e.g., by 5, 10, 20, 30, 40, 50, 75, 85, 90, 95 or 100%) the number of differentiated OPCs in injured optic nerves. In another embodiment, the agent is benztropine mesylate (Bzp), a M1/M3 muscarinic receptor antagonist (Deshmukh et al., Nature 502, 327-332 (2013)), clemastine (Clem), an antihistamine and anticholinergic agent, M1/M3 muscarinic receptor antagonist (Mei et al., Nat. Med. 20, 954-960 (2014)), Solifenacin (Sli), a M3 muscarinic receptor antagonist (Abiraman et al., J. Neurosci. 35, 3676-3688 (2015)), Bexarotene (Bex), a retinoid X receptor agonist (Natrajan et al., Brain 138, 3581-3597 (2015)), imidazole (Imi), an anti-cholesterol synthesis compound (Hubler et al., Nature 560, 372-376 (2018)); Ibudilast (Ibud), a clinically approved phosphodiesterase (PDE) inhibitor (Fox et al., N. Engl. J. Med. 379, 846-855 (2018)). Montelukast (Mon) and Pranlukast (Pra), two different GPR17 antagonists (Fumagalli et al., J. Biol. Chem. 286, 10593-10604 (2011), Marschallinger et al., Nat. Commun. 6. (2015); Ou et al., J. Neurosci. 36, 10560-10573 (2016)), rapamycin (Rap), which is an mTOR inhibitor, or thalidomide (a TNFα inhibitor).

By “alteration” is meant a change (increase or decrease) in the in myelination or in a marker (e.g., polynucleotide, polypeptide) associated with myelination as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease (e.g., a disease associated with a failure in myelination).

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, injury, or disorder that damages or interferes with the normal function of a cell, tissue, or organ. Examples of diseases include diseases associated with a failure in, loss of, or undesirable reduction in myelination, including but not limited to multiple sclerosis (MS), leukodystrophies, neurodegenerative disease, Alzheimer's disease, ALS, traumatic brain injury, and spinal cord injury. Examples of leukodystrophies include, but are not limited to, 18q Syndrome with Deficiency of Myelin Basic Protein, Adrenoleukodystrophy (ALD), Adrenomyeloneuropathy (AMN), Adult Onset Autosomal Dominant Leukodystrophy (ADLD), Adult Polyglucosan Body Disease, Aicardi-Goutieres Syndrome, Alexander Disease, Autosomal Dominant Diffuse Leukoencephalopathy with Neuroaxonal Spheroids (HDLS), AARS, AARS2, Canavan Disease, Cathepsin A-related arteriopathy with strokes and leukoencephalopathy (CARASAL), Cerebral Autosomal Dominant Arteriopathy with Subcortical Infarcts and Leukoencephalopathy (CADASIL), Cerebral Autosomal Recessive Arteriopathy with Subcortical Infarcts and Leukoencephalopathy (CARASIL), Cerebroretinal Micro-Angiography with Calcifications and Cysts, Cerebrotendinous Xanthomatosis (CTX), Childhood Ataxia with Central Nervous System Hypomyelination (CACH), ClC2-related leukoencephalopathy, Coates plus, Cockayne syndrome, Elongation of Very Long-Chain Fatty Acids-4 (ELOVL4; Pseudo-Sjogren-Larsson), Fatty Acid 2-Hydroxylase Deficiency, Fucosidosis, Congential Muscular Dystrophy, Globoid Cell Leukodystrophy (Krabbe Disease), GM1 Gangliosidosis, GM2 Gangliosidosis (Tay-Sachs Disease), Hypomyelination with Atrophy of the Basal Ganglia and Cerebellum (H-ABC), Hypomyelination, Hypogonadotropic, Hypogonadism and Hypodontia (4H Syndrome), Hypomyelination with Brainstem and Spinal Cord involvement and Leg Spasticity (HBSL), Hypomyelination with congenital cataract (HCC), Leukoencephalopathy with Brain Stem and Spinal Cord Involvement and Lactate Elevation (LBSL), Leukoencephalopathy with calcifications and cysts (LCC), Leukoencephalopathy with thalamus and brainstem involvement and high lactate (LTBL), Lipomembranous Osteodysplasia with Leukodystrophy (Nasu Disease), Metachromatic Leukodystrophy (MLD), Megalencephalic Leukodystrophy with subcortical Cysts (MLC), Mitochondrial leukodystrophies, Multiple sulfatase deficiency, Neuroaxonal Leukoencephalopathy with axonal spheroids (Hereditary diffuse leukoencephalopathy with spheroids (HDLS)), Neonatal Adrenoleukodystrophy (NALD), Oculodetatoldigital Dysplasia with Cerebral White Matter Abnormalities, Orthochromatic Leukodystrophy with Pigmented Glia, Ovarioleukodystrophy Syndrome, Pelizaeus Merzbacher Disease (X-linked spastic paraplegia), Pelizaeus Merzbacher like-disease (PMLD), RARS2-related hypomyelination, Refsum Disease, RNAse T2 deficient leukoencephalopathy, Sialic acid storage disorders (Salla disease, Infantile Sialic Acid Storage Disease and Intermediate form), Sjogren-Larsson Syndrome, SOX10-associated PCWH: peripheral demyelinating neuropathy, central dysmyelinating leukodystrophy, Waardenburg syndrome, and Hirschsprung disease, Vanishing White Matter Disease (VWM) or Childhood Ataxia with Diffuse Central Nervous System Hypomyelination (CACH), X-linked Adrenoleukodystrophy (X-ALD), and the Zellweger Spectrum (Zellweger Syndrome, Neonatal Adrenoleukodystrophy, and Infantile Refsum Disease).

By “effective amount” is meant the amount of a therapeutic composition required to ameliorate the symptoms of a disease, disorder, condition, or injury relative to an untreated patient. The effective amount of active compound(s) used to practice the present invention for therapeutic treatment of a disease, disorder, condition, or injury varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is an amount that increases myelination of a neuron, increases OPC proliferation, increases OPC number after injury, or promotes differentiation of OPCs to CC1− and Oligo1-positive cells.

The methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “insufficient myelination” is meant a reduced level of myelination of a target neuron relative to a level of myelination observed in a corresponding control neuron.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disease, disorder, condition, injury and/or symptoms associated therewith. It will be appreciated that, although not precluded, treating a disease, disorder, condition, or injury does not require that the disease, disorder, condition, injury or symptoms associated therewith be completely eliminated.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1P illustrate that increased proliferation and failed differentiation of OPCs in injured optic nerves.

FIG. 1A comprises representative electron microscopy images and quantifications of myelination percentage of axons in intact or injured optic nerves with regenerated axons. Most axons are myelinated in intact optic nerves and very rare regenerated axons have spontaneous myelination post optic nerve crush injury in adult mice. n=6 mice per group. Scale bar: 600 nm.

FIGS. 1B-1D present the scheme of experiments to assess OPC proliferation in both injured (ipsilateral) and intact (contralateral) optic nerves in PDGFRα-H2B-GFP reporter mice.

FIG. 1B is an illustration of injured ipsilateral and intact contralateral optic nerves. The arrow indicates crush injury site and the gray region indicates regions of interest that were analyzed.

FIG. 1C comprises immunofluorescent staining images in injured optic nerves at different time points after injury. n=3-8 mice per group. Scale bar: 100 μm.

FIG. 1D is a graph showing quantification of OPC numbers in injured optic nerves at different time points after injury. n=3-8 mice per group.

FIG. 1E comprises representative images of optic nerves (injured or their controls) stained with Oligo2 and/or GFP from PDGFRα-H2B-GPP reporter mice showing dynamic change of total OPC numbers at different time points after injury. Scale bar: 100 μm.

FIG. 1F comprises representative images of BrdU/Oligo2 double positive cells in injured optic nerves.

FIG. 1G is a graph showing the quantification of BrdU/Oligo2 double positive cells in injured optic nerves.

FIG. 1H is an illustration of different differentiation stages of OPCs and their respective markers. Scale bar: 50 μm.

FIG. 1I is a diagram of an experimental design for tracing the progenies of OPCs using PDGFRα-CreER/iRTM mice as shown in FIGS. 1J-1O.

FIG. 1J comprises representative images of CC1⁺ and RTM⁺ cells in injured and intact optic nerves.

FIG. 1K is a graph of CC1⁺ and RTM⁺ cell numbers in injured and intact optic nerves.

FIG. 1L is a graph showing the proportion of CC1⁺ and RTM⁺ cells in injured and intact optic nerves. In contrast to the intact (contralateral), the proportion of RTM⁺CC1⁺ were significantly less in injured nerves. n=6 mice per group.

FIG. 1M comprises representative images of three different populations (CC1⁻/Oligo1-N for un-differentiated OPCs, CC1⁺/Oligo1-N for pre-myelinating oligodendrocytes, and CC1⁺/Oligo1-C for mature myelinating oligodendrocytes) in injured and intact optic nerves. Arrows in the contralateral side indicate RTM⁺/CC1⁺/Oligo1-C, but those in ipsilateral side indicate RTM⁺/CC1⁻/Oligo1-N (un-differentiated cells). n=6 mice per group. Scale bar: 100 μm 10 μm.

FIG. 1N is a graph showing cell numbers of three different populations (CC1⁻/Oligo1-N (nuclear Oligo1) for undifferentiated OPCs, CC1+/Oligo1-N for pre-myelinating oligodendrocytes, and CC1⁺/Oligo1-C for mature myelinating oligodendrocytes) in injured and intact optic nerves. *, **, *** p<0.05, 0.01, 0.001, respectively.

FIG. 1O is a graph showing the proportions of three different populations (CC1⁻/Oligo1-N for undifferentiated OPCs, CC1+/Oligo1-N for pre-myelinating oligodendrocytes, and CC1⁺/Oligo1-C for mature myelinating oligodendrocytes) in injured and intact optic nerves. *, **, *** p<0.05, 0.01, 0.001, respectively.

FIG. 1P comprises representative images of injured optic nerves from PDGFRα-CreER/RTM mice, showing no overlapping between RTM and GFAP. Scale bar: 50 μm. *, **, *** p<0.05, 0.01, 0.001, respectively.

FIGS. 2A-2Z illustrate that GPR17 is an intrinsic blocker of early oligodendrocyte differentiation of OPCs in injured optic nerves.

FIG. 2A is a schematic of in vivo compound screening in wild type C57 mice. Daily BrdU injection between dpi4-10 was performed to label proliferating OPCs. Injured optic nerve from the mice received 4 week treatment of individual compounds were analyzed with indicated antibodies.

FIG. 2B comprises representative images of injured optic nerves stained with anti-CC1 and BrdU. n=4-13 mice per group.

FIG. 2C is a graph that quantifies injured optic nerves stained with anti-CC1 and BrdU. n=4-13 mice per group.

FIG. 2D is a schematic of an experimental design for studies using Montelukast shown in FIGS. 2E-2G in PDGFRα-CreER:RTM mice. n=6 mice per group.

FIG. 2E comprises representative images of injured or intact optic nerves stained with antibodies against Oligo1, CC1, RTM, and DAPI anti-CC1 and BrdU (of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of RTM⁺ cells.

FIG. 2F is a graph showing the densities of different populations (CC1⁻/Oligo1-N for un-differentiated OPCs, CC1⁺/Oligo1-N for pre-myelinating oligodendrocytes, and CC1⁺/Oligo1-C for mature myelinating oligodendrocytes) of RTM+ cells in injured or intact optic nerves stained with antibodies against Oligo1, CC1, RTM, and DAPI anti-CC1 and BrdU.

FIG. 2G is a graph showing the proportions of different populations (CC1−/Oligo1-N for un-differentiated OPCs, CC1+/Oligo1-N for pre-myelinating oligodendrocytes, and CC1+/Oligo1-C for mature myelinating oligodendrocytes) of RTM+ cells.

FIG. 2H comprises representative in situ hybridization images of injured optic nerves showing injury-induced Gpr17 expression. Scale bar: 100 μm.

FIG. 2I is a graph quantifying the optic nerves showing injury-induced Gpr17 expression in FIG. 2H. n=6 mice per group.

FIGS. 2J-2L show that Gpr17 knockout did not affect OPC proliferation.

FIG. 2J comprises representative images of injured optic nerves of GPR17 knockout mice and their controls that received daily BrdU injection between 4-10 dpi, stained with GFP (GPR17), BrdU and/or Oligo2. Scale bar: 200 μm.

FIG. 2K is a graph quantifying injured optic nerves of FIG. 2J stained with GFP (GPR17), BrdU, and Oligo2.

FIG. 2L is a graph quantifying injured optic nerves of FIG. 2J stained with BrdU and Oligo2.

FIG. 2M-2O illustrate an OPC differentiation analysis with antibodies against GFP (GPR17) or CC1 in injured optic nerves of GPR17 knockout mice.

FIG. 2M comprises representative images of injured optic nerves taken from GPR17 knockouts or their controls at 28 days after injury.

FIG. 2N is a graph quantifying the densities of GFP⁺CC1⁺ cells from GPR17 knockout mice having injured optic nerves and their controls.

FIG. 2O is a graph quantifying the proportion of CC1⁺ among GFP⁺ cells from GPR17 knockout mice having injured optic nerves and their controls.

FIG. 2P comprises representative images of injured optic nerves from GPR17 knockout and their controls stained with GFP (GPR17) and CC1. n=6 mice per group. Scale bar: 200 μm.

FIG. 2Q is a graph quantifying the proportion of CC1⁺ cells among GFP⁺ cells in GPR17 knockout mice having injured optic nerves and their controls.

FIG. 2R is a graph quantifying the densities of GFP⁺CC1⁺ cells from GPR17 knockout mice having injured optic nerves and their controls. FIGS. 2Q and 2R show that GPR17 knockout significantly increased CC1⁺ cells in injured but not intact optic nerves.

FIGS. 2S-2U OPC illustrates a differentiation analysis with antibodies against GFP (GPR17), CC1, Oligo1, DAPI in injured optic nerves (28 days after injury) of GPR17 knockout mice.

FIG. 2S comprises representative images of injured optic nerves from GPR17 knockout mice and their controls stained with antibodies against Oligo1, CC1, RTM, and DAPI anti-CC1 and BrdU. Scale bar: 50 μm.

FIG. 2T is a graph quantifying the densities of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of GFP⁺ cells in GPR17 knockout mice having injured optic nerves and their controls. n=6 mice per group. *, **, *** p<0.05, 0.01, 0.001, respectively.

FIG. 2U is a graph quantifying the proportions of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of GFP⁺ cells in GPR17 knockout mice having injured optic nerves and their controls. n=6 mice per group. *, **, *** p<0.05, 0.01, 0.001, respectively.

FIGS. 2V-2X illustrate an OPC differentiation analysis with antibodies against GFP (GPR17), CC1, Oligo1, DAPI in injured optic nerves (7 days after injury) of GPR17 knockout mice.

FIG. 2V comprises representative images of injured optic nerves from GPR17 knockout mice and their controls stained with antibodies against Oligo1, CC1, RTM, and DAPI anti-CC1 and BrdU and quantification results of the densities (J) or proportions (K) of different populations (CC1⁻/Oligo1-N for un-differentiated OPCs, CC1⁺/Oligo1-N for pre-myelinating oligodendrocytes, and CC1⁺/Oligo1-C for mature myelinating oligodendrocytes) of GFP⁺ cells. Scale bar: 100 μm.

FIG. 2W is a graph quantifying the densities of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of GFP⁺ cells from GPR17 knockout mice having injured optic nerves and their controls. n=6 mice per group.

FIG. 2X is a graph quantifying the proportions of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of GFP⁺ cells from GPR17 knockout mice having injured optic nerves and their controls. n=6 mice per group.

FIG. 2Y comprises representative images of injured optic nerves from GPR17 knockout mice and their controls that received daily BrdU injection from 4-10 dpi, stained with GFP (GPR17) and CC1 (L).

FIG. 2Z is a graph quantifying the proportion of CC1⁺ cells among BrdU⁺ cells from GPR17 knockout mice with injured optic nerves and their controls. n=6 mice per group. *, **, *** p<0.05, 0.01, 0.001, respectively.

FIGS. 3A-3G illustrate that activated microglia in injured optic nerves were depleted by PLX3397 treatment.

FIG. 3A comprises representative images of optic nerves, from adult mice 4 weeks after unilateral optic nerve crush injury, stained with antibodies against GFAP, CD68 or P2Y12. Scale bar: 50 μm.

FIG. 3B is a graph showing the quantification of GFAP immunoreactivity signal. n=10 mice per group.

FIG. 3C is a graph showing the quantification of P2Y12 immunoreactivity signal. n=10 mice per group.

FIG. 3D is a graph showing the quantification of CD68 immunoreactivity signal. n=10 mice per group.

FIG. 3E comprises images showing sustained activated microglia in the entire optic nerves distal to the lesion, taken from adult mice at 6 weeks post injury. Scale bar: 500 μm.

FIG. 3F is a graph quantifying the CD68 immunoreactivity signal. n=10 mice per group.

FIG. 3G comprises representative images of optic nerves, from adult mice 2 weeks after unilateral optic nerve crush injury and with or without PLX3397 treatment, stained with antibodies against CD68. Scale bar: 40 μm.

FIGS. 3H-30 illustrate that microglia are required for OPC proliferation but detrimental for their maturation.

FIG. 3H is a graph quantifying the CD68 immunoreactivity signal. 6=10 mice per group. Scale bar: 40 μm. *, **, *** p<0.05, 0.01, 0.001, respectively.

FIG. 3I is a schematic of experiments to assess OPC proliferation with the treatment of PLX3397 (PLX) or its vehicle control (Vec) in PDGFRα-H2B-GFP mice. PLX treatment was applied for 14 days (7 days before and after injury), and BrdU was injected 48 hours before termination.

FIG. 3J comprises representative images of injured optic nerves stained with GFP, Oligo2, or BrdU. Scale bar: 100 μm

FIG. 3K is a graph quantifying the densities of GFP⁺Oligo2⁺BrdU⁺ cells. n=6 mice per group.

FIG. 3L is a schematic of an experimental design for analyzing the effects of delayed PLX3397 treatment on OPC differentiation in injured optic nerves for the results shown in FIGS. 3M-O in PDGFRα-CreER:RTM mice.

FIG. 3M comprises representative images of injured or intact optic nerves stained with antibodies against Oligo1, CC1, RTM, and DAPI anti-CC1 and BrdU Scale bar: 100 μm.

FIG. 3N is a graph quantifying the densities of different populations (CC1⁻/Oligo1-N for un-differentiated OPCs, CC1⁺/Oligo1-N for pre-myelinating oligodendrocytes, and CC1⁺/Oligo1-C for mature myelinating oligodendrocytes) of RTM⁺ cells. n=6 mice per group. *, **, *** p<0.05, 0.01, 0.001, respectively.

FIG. 3O is a graph quantifying the proportions of different populations (CC1⁻/Oligo1-N for un-differentiated OPCs, CC1⁺/Oligo1-N for pre-myelinating oligodendrocytes, and CC1⁺/Oligo1-C for mature myelinating oligodendrocytes) of RTM⁺ cells. n=6 mice per group. *, **, *** p<0.05, 0.01, 0.001, respectively.

FIGS. 4A-4I illustrate that combinatorial treatment of Montelukast and PLX3397 leads to robust myelination of regenerated axons in injured optic nerves of adult mice FIG. 4A comprises representative images of injured optic nerves, from adult PDGFRα-CreER:RTM mice that received Montelukast and PLX3397 treatment, stained with antibodies against Oligo1, CC1, RTM, and DAPI anti-CC1 and BrdU. The samples were collected at the end of week 4 after injury. Scale bar: 20 μm (A).

FIG. 4B is a graph quantifying the densities of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of RTM⁺ cells. n=6 mice per group.

FIG. 4C is a graph quantifying the proportions of different populations (CC1⁻/Oligo1-N for undifferentiated OPCs, CC1⁺/Oligo1-N for pre-myelinating oligodendrocytes, and CC1⁺/Oligo1-C for mature myelinating oligodendrocytes) of RTM⁺ cells. n=6 mice per group.

FIG. 4D-H illustrate the transmission electron microscope imaging results of myelination of regenerated axons of injured optic nerves (at 4 weeks after injury) from the mice with the treatment of Montelukast and/or PLX3397.

FIG. 4D is a low magnification image of coronal sections of crushed optic nerves from each treatment group. Scale bar: 2 μm.

FIG. 4E is an enlarged image of ongoing myelination on regenerated axons from the combined treatment group. The thin layers of myelin and the large inner tongue suggested ongoing new myelination. Scale bar: 500 nm.

FIG. 4F A montage of images from longitudinal sections of a crushed optic nerve with the combined treatment. A complete internode is indicated by “x”. Arrows indicate the positions of neighboring Nodes of Ranvier. Scale bar: 1400 nm.

FIG. 4G is an enlarged image of half of Nodes of Ranvier on regenerated axons is shown in G. (H) Quantifications of panel D. n=4 mice per group. Scale bar: 200 nm.

FIG. 4H is a graph quantifying the percentages of myelinated axons observed in FIG. 4D.

FIG. 4I comprises representative images of injured optic nerves with the combined treatments stained with nodes of Ranvier markers, Caspr, AnkG and sodium channel NaV1.6. Scale bar: 3.5 μm.

FIGS. 4J-4L illustrate that better preserved regenerated axons in the mice with the combinatorial treatment of Montelukast and PLX3397.

FIG. 4J comprises representative images of CTB-labeled regenerating axons in injured optic nerves (42 dpi) from wild type mice that received intravitreal injection with AAV2/2-CNTF/IGF/OPN, followed by optic nerve crush and with or without Montelukast and PLX3397 treatment. Scale bar: 750 μm.

FIG. 4K is a graph quantifying the area of regenerated axons relative to the time post injury. n=4 mice per group.

FIG. 4L is a graph quantifying the intensity of the signal associated with regenerating axons relative to the distance from the crush site. n=4 mice per group.

FIGS. 5A-5E illustrate inhibition of TNFα on cell populations after optic nerve crush.

FIG. 5A comprises a Venn diagraph and a heatmap showing that TNFα is upregulated after optic nerve crush.

FIG. 5B comprises fluorescence images showing adult optic nerve after crush probed for TNFα.

FIG. 5C comprises fluorescence images and graphs showing a robust increase of CC1⁺ oligodendrocytes at distal region near crush site in TNFR1 KO mice.

FIG. 5D comprises fluorescence images that show a robust increase of BrdU⁺/CC1⁺/Olig2⁺ cells at the distal region of an axon in mice treated with thalidomide, a TNFα inhibitor. TNFi denotes thalidomide

FIG. 5E comprises schematics and images showing comparisons of mice treated with vehicle alone and mice treated with thalidomide.

### DETAILED DESCRIPTION OF THE INVENTION

The invention features compositions and methods that are useful for promoting or increasing myelination, especially in cases of nerve injury or demyelination resulting from disease.

The invention is based, at least in part, on the discovery that inhibiting GPR17 and ablating activated microglia resulted in robust myelination of regenerated axons.

Myelination facilitates axon conduction, enabling efficient communication among different parts of the nervous system. Manipulations that elevate the intrinsic regenerative ability of neurons result in robust axon regeneration after optic nerve injury, but these regenerating axons do not undergo spontaneous myelination. The underlying mechanisms for such myelination failure remains elusive.

As demonstrated herein, oligodendrocyte precursor cells (OPCs) in the optic nerve undergo a transient proliferation but fail to differentiate into mature myelination-competent oligodendrocytes in an optic nerve injury model in adult mice, which is reminiscent of progressive multiple sclerosis. Mechanistically, both OPC-intrinsic GPR17 and chronically activated microglia inhibit different stages of OPC differentiation. Importantly, inhibiting both GPR17 and microglia led to robust myelination of regenerated axons. In addition to revealing regulatory mechanisms of stage-dependent OPC differentiation, the results presented indicated that the agents described herein provide for robust de novo myelination even in the presence of chronic inflammatory conditions in the adult CNS.

### Myelination of Neurons

Myelination of regenerated or demyelinated axons by mature myelinating oligodendrocytes is required to have a properly functioning nerve. As oligodendrocytes are responsible for myelination, eliciting expansion and maturation of oligodendrocyte precursor cells (OPCs) represents a significant improvement in treating nerve injury or demyelination resulting from disease. Undifferentiated, early differentiated, and mature myelinating oligodendrocytes can be distinguished based on the cells CC1 and Oligo1 expression profile. For example, undifferentiated oligodendrocytes are characterized as being CC1-negative cells having Oligo1 localized to the nucleus. Early differentiated oligodendrocytes are characterized as CC1-positive cells having Oligo1 localized to the nucleus. Mature myelinating oligodendrocytes are characterized as CC1-positive cells having cytoplasmic Oligo1. In response to a neural injury, OPCs are expanded. In some embodiments, maturation of an undifferentiated OPC to an early differentiated OPC can be promoted by contacting the cell with a GPR17 antagonist or inhibitor. In some embodiments, the GPR17 antagonist or inhibitor can be benztropine mesylate, a M1/M3 muscarinic receptor antagonist; clemastine, an antihistamine and anticholinergic agent and M1/M3 muscarinic receptor antagonist; solifenacin, a M3 muscarinic receptor antagonist; bexarotene, a retinoid X receptor agonist; imidazole, an anti-cholesterol synthesis compound; ibudilast, a clinically approved phosphodiesterase (PDE) inhibitor; montelukast or pranlukast, or thalidomide.

Early differentiated OPCs can be further differentiated into mature myelinating oligodendrocytes by removing microglia in the surrounding environment. Ablation agents are known in the art that effectively remove ablate microglia. In some embodiments of the present invention, contact microglia with an inhibitor or ablation agent provides an environment that is conducive to differentiating the early differentiated oligodendrocyte into a mature myelinating oligodendrocyte.

Agents of the Invention Agents described herein, including small compounds, are useful for increasing myelination, increasing OPC proliferation or differentiation, or increasing OPC number. In one embodiment, an agent of the invention is Pexidartinib, also termed PLX3397, which is a small molecule having multi-kinase inhibitory activity. Pexidartinib (CAS Registry No. 1029044-16-3; C₂₀—H₁₅—Cl—F₃—N₅ has the following structure.

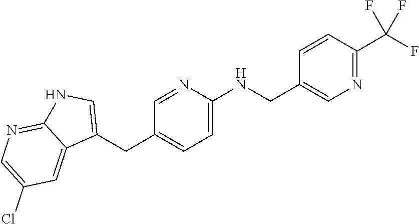 [SMILES: FC(F)(F)c1ccc(CNc2ccc(Cc3c[nH]c4ncc(Cl)cc34)cn2)cn1]

In another embodiment, the agent of the invention is bexarotene (e.g., 100 mg/kg, p.o.), which is a small molecule having retinoid X receptors binding and activation activity. Bexarotene (CAS Registry No. 153559-49-0; C₂₄H₂₈O₂), has the following structure:

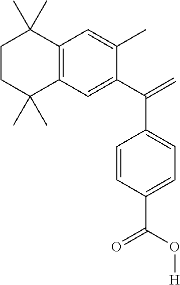 [SMILES: C=C(c1ccc(C(=O)O)cc1)c1cc2c(cc1C)C(C)(C)CCC2(C)C]

In another embodiment, the agent of the invention is benztropine mesylate (e.g., 10 mg/kg, i.p.), which is a small molecule central muscarinic antagonist having dopamine uptake inhibitory activity. Benztropine mesylate (CAS Registry No. 132-17-2; C₂₂H₂₉NO₄S) has the following structure:

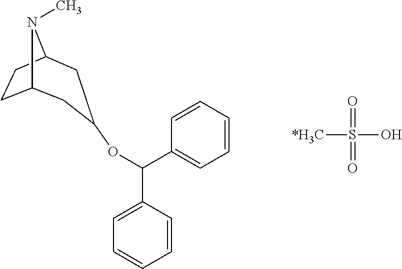

In another embodiment, the agent of the invention is clemastine fumarate (e.g., 10 mg/kg, p.o.), which is a small molecule having anticholinergic, sedative, and histamine H1 antagonistic properties. Clemastine fumarate (CAS Registry No. 14976-57-9; C₂₅H₃₀ClNO₅) has the following structure:

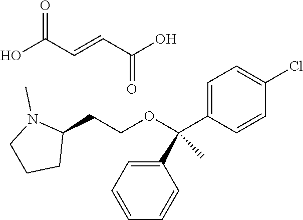

In another embodiment, the agent of the invention is ibudilast (e.g., 10 mg/kg, i.p.), which is a small molecule having cyclic nucleotide phosphodiesterase inhibitory activity. Ibudliast (CAS Registry No. 50847-11-5; C₁₄H₁₈N₂O) has the following structure:

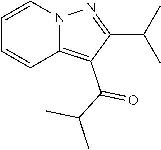 [SMILES: CC(C)C(=O)c1c(C(C)C)nn2ccccc12]

In another embodiment, the agent of the invention is imidazole (e.g., 10 mg/kg, i.p.), which is a base and an excellent nucleophile. Imidazole (CAS Registry No. 288-32-4, C₃H₄N₂) has the following structure:

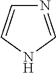

In another embodiment, the agent of the invention is montelukast (e.g., 25 mg/kg, p.o.), which is a leukotriene receptor (e.g., GRP17) antagonist. Montelukast (CAS Registry No. 158966-92-8; C₃₅H₃₆ClNO₃S) has the following structure:

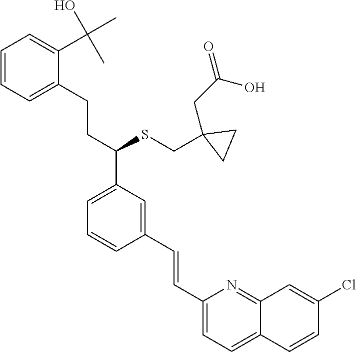 [SMILES: CC(C)(O)c1ccccc1CC[C@@H](SCC1(CC(=O)O)CC1)c1cccc(/C=C/c2ccc3ccc(Cl)cc3n2)c1]

In another embodiment, the agent of the invention is pranlukast (e.g., 0.5 mg/kg, i.p.), which is a leukotriene receptor (e.g., GRP17) antagonist. Pranlukast (CAS Registry No. 103177-37-3; C₂₇H₂₃N₅O₄) has the following structure:

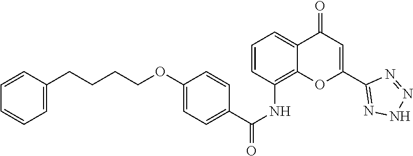 [SMILES: O=C(Nc1cccc2c(=O)cc(-c3nn[nH]n3)oc12)c1ccc(OCCCCc2ccccc2)cc1]

In another embodiment, the agent of the invention is rapamycin (e.g., 6 mg/kg, i.p.), which has mTOR inhibitory activity. Rapamycin (CAS Registry No. 53123-88-9; C₅₁H₇₉NO₁₃) has the following structure:

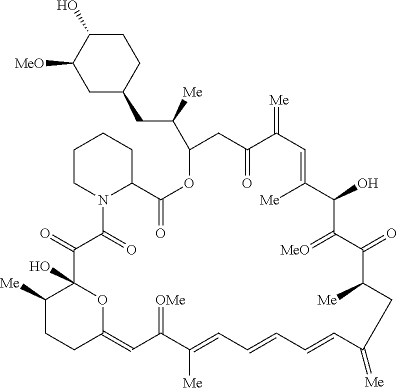

In another embodiment, the agent of the invention is solifenacin succinate (e.g., 20 mg/kg, i.p.), which is a small molecule having a anticholinergic and antispasmodic activity. Solifenacin succinate (CAS Registry No. 242478-38-2; C₂₇H₃₂N₂O₆) has the following structure:

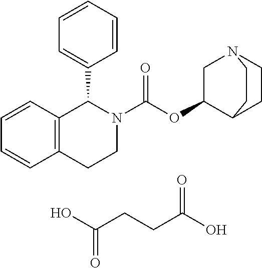 [SMILES: O=C(O[C@H]1CN2CCC1CC2)N1CCc2ccccc2[C@@H]1c1ccccc1 ; O=C(O)CCC(=O)O]

In another embodiment, the agent of the invention is thalidomide (e.g., 50 mg/kg, i.p.), which is a small molecule that inhibits the production of tumor necrosis factor alpha (TNFα). Thalidomide (CAS Registry No. 50-35-1; C₁₃H₁₀N₂O₄) has the following structure:

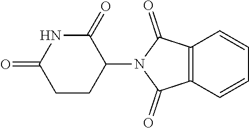 [SMILES: O=C1CCC(N2C(=O)c3ccccc3C2=O)C(=O)N1]

### Methods of Treatment

The present invention provides methods of treating disease, disorders, or injuries characterized by unmyelinated or demyelinated neurons, or symptoms thereof, that comprise administering a therapeutically effective amount of a pharmaceutical composition comprising an agent described herein (e.g., G Protein-Coupled Receptor 17 (GPR17) antagonist, a microglia inhibitor or ablation agent). In some embodiments, the disease is characterized by demyelination of nerves. In some embodiments, the disease is a neurodegenerative disease, such as multiple sclerosis or Alzheimer's. In some embodiments, the injury to be treated is a traumatic brain injury.

In some embodiments, the agent is benztropine mesylate, clemastine, solifenacin, bexarotene, imidazole, ibudilast, montelukast, pranlukast, or thalidomide. In some embodiments, the microglia inhibitor or ablation agent is PLX3397. Thus, one embodiment is a method of treating a subject suffering from or susceptible to a disease or disorder or symptom thereof. The method includes the step of administering to the mammal a therapeutic amount of an agent disclosed herein sufficient to treat the disease or disorder or symptom thereof, under conditions such that the disease or disorder is treated.

Identifying a subject in need of such treatment can be in the judgment of a subject or a health care professional and can be subjective (e.g., opinion) or objective (e.g., measurable by a test or diagnostic method). Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like). The compounds herein may be also used in the treatment of any other disorders in which myelination deficiency or loss may be implicated.

### Compositions

A GPR17 antagonist and a microglia inhibitor or ablation agent, when administered as a combination, are useful for the treatment of diseases, disorders, or injuries characterized by insufficient myelination of neurons. In some embodiments, the GPR17 antagonist is montelukast, or pranlukast. In some embodiments the microglia inhibitor or ablation agent is PLX3397. In certain embodiments, a GPR17 antagonist and microglia inhibitor or ablation agent combination therapy can increase myelination of a target neuron by at least 10%, 25%, 50%, 75%, or even 100%.

Pharmaceutically acceptable salts of the GPR17 antagonist and the microglia inhibitor or ablation agent or both are contemplated herein for increasing myelination of a target neuron. The term “pharmaceutically acceptable salt” also refers to a salt prepared from a GPR17 antagonist or a microglia inhibitor or ablation agent, wherein the GPR17 antagonist or the microglia inhibitor or ablation agent has an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a GPR17 antagonist and/or a microglia inhibitor or ablation agent having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

### Pharmaceutical Therapeutics

For therapeutic uses, compositions comprising an agent described herein may be administered systemically. Preferable routes of administration include, for example, oral administration or subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. In one embodiment, a GPR17 antagonist, a microglia inhibitor or ablation agent, or both can be formulated in a pharmaceutically-acceptable buffer such as physiological saline. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of disease or disorder or injury characterized by insufficient myelination. Generally, amounts will be in the range of those used for other agents used in the treatment of other conditions or diseases or injuries characterized by insufficient myelination. In some embodiments, a composition comprising an agent described herein (e.g., a GPR17 antagonist and/or a microglia inhibitor or ablation agent, is administered at a dosage that is effective at increasing myelination of a target neuron. In other embodiments, a composition comprising a GPR17 antagonist and a composition comprising a microglia inhibitor or ablation agent are administered at dosages that are effective at increasing myelination of a target neuron. Effectiveness of the administration can be determined by a method known to one skilled in the art, or using any assay that measures myelination of neurons.

### Formulation of Pharmaceutical Compositions

The administration of a composition comprising an agent described herein (e.g., GPR17 antagonist, a microglia inhibitor or ablation agent, or both) for the treatment of diseases or disorders or injuries characterized by insufficient myelination of a target neuron may be by any suitable means that results in a concentration of the therapeutic that, combined with other components, is effective in increasing or stabilizing the myelination of a target neuron. The composition may be contained in any appropriate amount in any suitable carrier substance, and is generally present in an amount of 1-95% by weight of the total weight of the composition. The composition may be provided in a dosage form that is suitable for oral administration. In some embodiments, the composition may be provided in a dosage form that is suitable for a parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of the agent used in mice, as a skilled artisan recognizes it is routine in the art to modify the dosage for humans compared to animal models. Dosages may be determined based on dosages for the effective treatment of conditions or injuries known in the art that are characterized by insufficient myelination of a target neuron. In certain embodiments it is envisioned that the dosages of an agent described herein is between about 0.1 mg and about 200 mg per day, between about 0.1 mg and about 190 mg per day, about 0.1 mg and about 180 mg per day, about 0.1 mg and about 170 mg per day, about 0.1 mg and about 160 mg per day, about 0.1 mg and about 150 mg per day, about 0.1 mg and about 140 mg per day, about 0.1 mg and about 130 mg per day, about 0.1 mg and about 120 mg per day, about 0.1 mg and about 110 mg per day, about 0.1 mg and about 100 mg per day, about 0.1 mg and about 90 mg per day, about 0.1 mg and about 80 mg per day, about 0.1 mg and about 70 mg per day, about 0.1 mg and about 60 mg per day, about 0.1 mg and about 50 mg per day, about 0.1 mg and about 40 mg per day, about 0.1 mg and about 30 mg per day, about 0.1 mg and about 20 mg per day, about 0.1 mg and about 10 mg per day, about 0.1 mg and about 5 mg per day, about 0.1 mg and about 1 mg per day. In some embodiments, the dosage amounts of a GPR17 antagonist is between about 0.5 mg and about 200 mg per day, about 1 mg and about 200 mg per day, about 10 mg and about 200 mg per day, about 20 mg and about 200 mg per day, between about 30 mg and about 200 mg per day, between about 40 mg and about 200 mg per day, between about 50 mg and about 200 mg per day, between about 60 mg and about 200 mg per day, between about 70 mg and about 200 mg per day, between about 80 mg and about 200 mg per day, between about 90 mg and about 200 mg per day, between about 100 mg and about 200 mg per day, between about 110 mg and about 200 mg per day, between about 120 mg and about 200 mg per day, between about 130 mg and about 200 mg per day, between about 140 mg and about 200 mg per day, between about 150 mg and about 200 mg per day, between about 160 mg and about 200 mg per day, between about 170 mg and about 200 mg per day, between about 180 mg and about 200 mg per day, or between about 190 mg and about 200 mg per day.

In some embodiments, the dosage of a microglia inhibitor or ablation agent is between about 250 and about 350 mg per day. In some embodiments, the dosage of the microglia inhibitor or ablation agent is between about 250 mg and about 325 mg per day, between about 250 mg and about 300 mg per day, or between about 250 mg and about 275 mg per day. In some embodiments, the dosage amount of a microglia inhibitor or ablation agent is between about 275 mg and about 350 mg per day, between about 300 mg and about 350 mg per day, or between about 325 mg and about 350 mg per day.

In some embodiments, the agent is bexarotene and has a dosage amount of about 50 to about 150 mg/kg. In some embodiments, the agent is benztropine mesylate and has a dosage amount of about 5 to about 15 mg/kg. In some embodiments, the agent is clemastin fumarate and has a dosage amount of about 5 to about 15 mg/kg. In some embodiments, the agent is ibudilast and has a dosage amount of about 5 to about 15 mg/kg. In some embodiments, the agent is imidazole and has a dosage amount of about 5 to about 15 mg/kg. In some embodiments, the agent is montelukast and has a dosage amount of about 10 to about 40 mg/kg. In some embodiments, the agent is pranlukast and has a dosage amount of about 0.1 to about 1.0 mg/kg. In some embodiments, the agent is rapamycin and has a dosage amount of about 3 to about 9 mg/kg. In some embodiments, agent is solifenacin succinate and has a dosage amount of about 10 to about 30 mg/kg. In some embodiments, the agent is thalidomide and has a dosage amount of about 25 to about 75 mg/kg. In some embodiments, the agent is pexidartinib (PLX 3397) and has a dosage amount of about 225 to about 350 mg/kg per day. Of course, the dosage amounts may be adjusted upward or downward, as is routinely done in such treatment protocols, depending on the results of the initial clinical trials and the needs of a particular patient.

Pharmaceutical compositions according to the presently disclosed embodiments may be formulated to release the active compound (e.g., a GPR17 antagonist and a microglia ablation agent,) substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition near intended targeted cells (e.g., brain cells); (v) formulations that allow for convenient dosing, such that doses are administered, for example, orally once or twice per day; and (vi) formulations that target calcium channels and angiotensin receptors by using carriers or chemical derivatives to deliver the therapeutic agent to a particular cell type (e.g., brain cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agents that increase myelination of a target neuron, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the presently disclosed embodiments may be in the form suitable for sterile injection. To prepare such a composition, the GPR17 antagonist and/or the microglia ablation agent, is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drugs may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredients (e.g., a GPR17 antagonist and a microglia ablation agent,) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets are uncoated in some embodiments and coated in other embodiments. The tablets can be coated by known techniques, optionally to delay disintegration and absorption in the gastrointestinal tract and thereby provide a sustained action over a longer period. The coating can be adapted to release the active drug or drugs in a predetermined pattern (e.g., in order to achieve a controlled release formulation) or adapted not to release the active drug until after passage of the stomach (enteric coating). The coating, in some embodiments, is a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate can be employed.

The solid tablet compositions include, in some embodiments, a coating adapted to protect the composition from unwanted chemical changes, (e.g., chemical degradation prior to the release of the agent). In some embodiments, the coating is applied on the solid dosage form in a similar manner as that described in Encyclopedia of Pharmaceutical Technology, supra.

In one embodiment, a GPR17 antagonist and a microglia ablation agent, can be mixed together in the tablet or partitioned. In one example, the GPR17 antagonist is contained on the inside of the tablet, and the microglia ablation agent is on the outside, such that a substantial portion of the microglia ablation agent is released prior to the release of the GPR17 antagonist. In some embodiments, the microglia ablation agent is contained on the inside of the tablet and the GPR17 antagonist on the outside.

Formulations for oral use include chewable tablets or hard gelatin capsules, wherein the active ingredients (i.e., the GPR17 antagonist and the microglia ablation agent) are mixed with an inert solid diluent (e.g., potato starch, lactose, microcrystalline cellulose, calcium carbonate, calcium phosphate, or kaolin), or as soft gelatin capsules, wherein the active ingredients are mixed with water or an oil medium, for example, peanut oil, liquid paraffin, or olive oil. Powders and granulates are prepared in some embodiments using the ingredients mentioned above under tablets and capsules in a conventional manner using, e.g., a mixer, a fluid bed apparatus, or a spray drying equipment.

### Controlled Release Oral Dosage Forms

Controlled release compositions of an agent, for oral use may, e.g., be constructed to release the agent, by controlling the dissolution and/or the diffusion of the active substance. Dissolution or diffusion controlled release can be achieved by appropriate coating of a tablet, capsule, pellet, or granulate formulation of compounds, or by incorporating the composition comprising the agent, into an appropriate matrix. A controlled release coating includes, in some embodiments, one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material can also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing an agent is, in some embodiments, in the form of a buoyant tablet or capsule (i.e., a tablet or capsule that, upon oral administration, floats on top of the gastric content for a certain period of time). A buoyant tablet formulation of the composition can be prepared by granulating a mixture of a GPR17 antagonist and a microglia ablation agent, with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

The presently disclosed embodiments provide methods of treating diseases, disorders, or injuries characterized by insufficient myelination that comprise administering a therapeutically effective amount of a pharmaceutical composition comprising a GPR17 antagonist and a microglia ablation agent, to a subject (e.g., a mammal such as a human). The method includes the step of administering to the subject a therapeutic amount of an amount of a GPR17 antagonist and a microglia ablation agent, sufficient to treat the disease, condition, disorder, injury or symptom thereof, under conditions such that the disease, condition, disorder, or injury or symptom thereof is treated. The therapeutic methods include prophylactic treatment. In some embodiments, the subject is a mammal, particularly a human suffering from, having, susceptible to, or at risk for disease or disorder that is characterized by insufficient myelination of a target neuron.

### Combination Therapies

Optionally, a GPR17 antagonist and a microglia ablation agent, may be administered in combination with any other standard treatment for a disease, disorder, condition, or injury characterized by insufficient myelination; such methods are known to the skilled artisan and described in Remington's Pharmaceutical Sciences by E. W. Martin.

### Kits or Pharmaceutical Systems

The present compositions may be assembled into kits or pharmaceutical systems for use in increasing myelination of a target neuron. Kits or pharmaceutical systems comprise a carrier means, such as a box, carton, tube or the like, having in close confinement therein one or more container means, such as vials, tubes, ampoules, bottles and the like. The kits or pharmaceutical systems may also comprise associated instructions for using the agents of the presently disclosed embodiments. In some embodiments, kits include a GPR17 antagonist and a microglia ablation agent. In some embodiments, the GPR17 antagonist is montelukast, or pranlukast. In some embodiments, the microglia ablation agent is PLX3397.

### Identification of Compounds and Compositions

The present invention provides methods for identifying compounds and compositions that can be used to differentiate oligodendrocyte precursor cells (OPCs) into early differentiated oligodendrocytes or that can be used to differentiated early differentiated oligodendrocytes into mature myelinating oligodendrocytes. Neuronal injury and diseases that are characterized by insufficient myelination (e.g., multiple sclerosis) can be studied using an optical nerve crush mouse model. This model involves damaging the optic nerve as described below or as is known in the art. Candidate compounds are administered to the animal. In some embodiments, oligodendrocyte differentiation is observed as a measurement of the effectiveness of the compound(s) or composition(s) for differentiating OPCs or early differentiated oligodendrocytes. In some embodiments, myelination of regenerated neurons is monitored to determine effectiveness of the candidate compounds.

This method of identification is particular appropriate for identifying candidate treatments for multiple sclerosis. Optic nerve injury, or optic crush, is a superior model for multiple sclerosis relative to traditional methods involving injecting androgens into mice to trigger demyelination. While the animals receiving androgen injections enjoy spontaneous myelination after the androgen administration is stopped, this is not the case with optic crush. Mice receiving optic crush injury exhibit increased inflammation and no spontaneous remyelination, both of which are in common with multiple sclerosis, leukodystrophies, neurodegenerative Alzheimer's disease, and central nervous system injuries (e.g., traumatic brain injury, spinal cord injury) associated with myelination failure

The practice of the presently disclosed embodiments employ, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Injury-Induced OPCs Proliferation

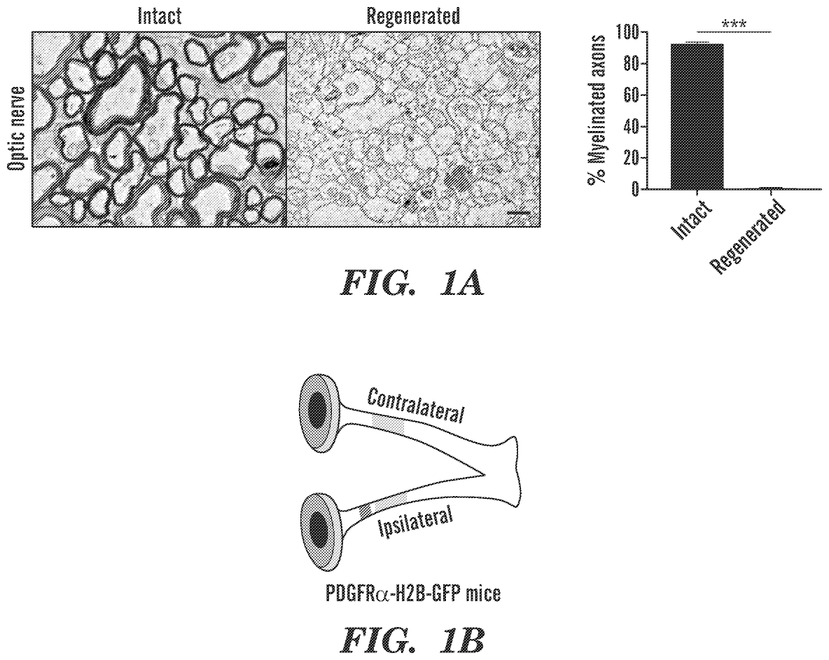

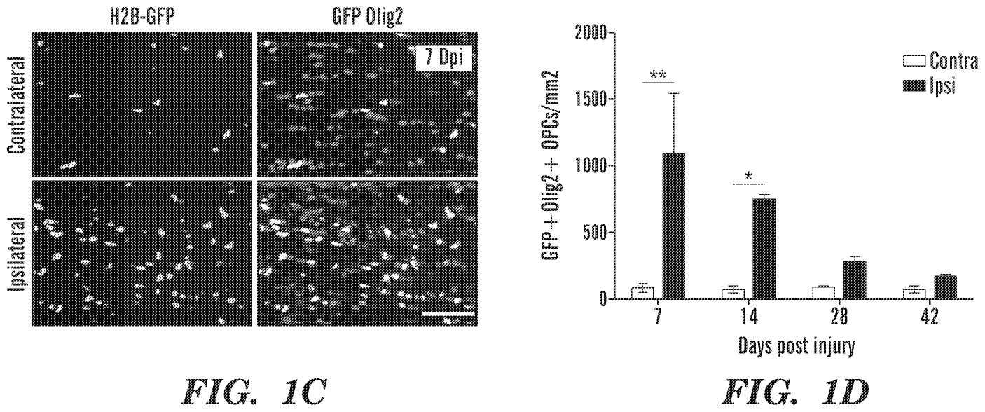

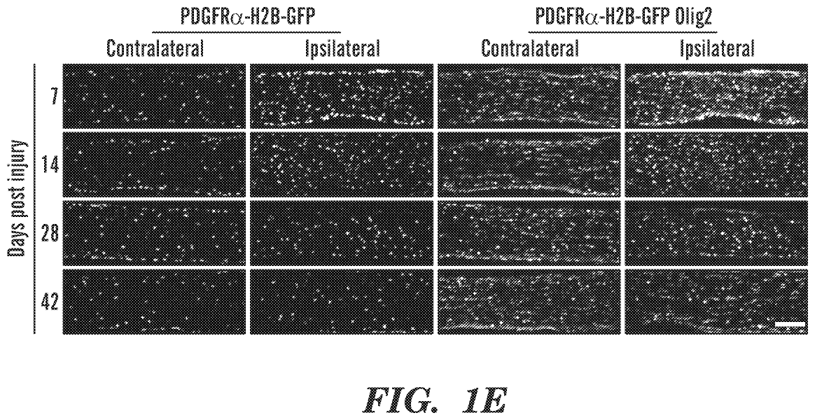

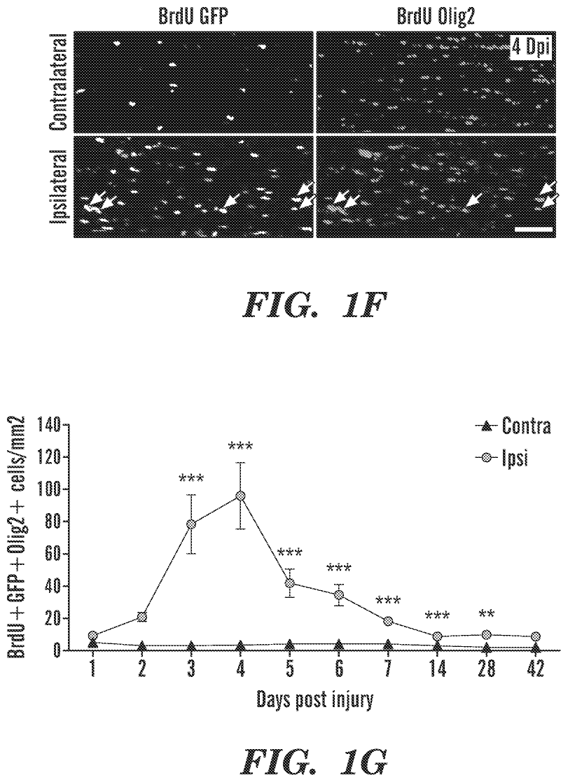

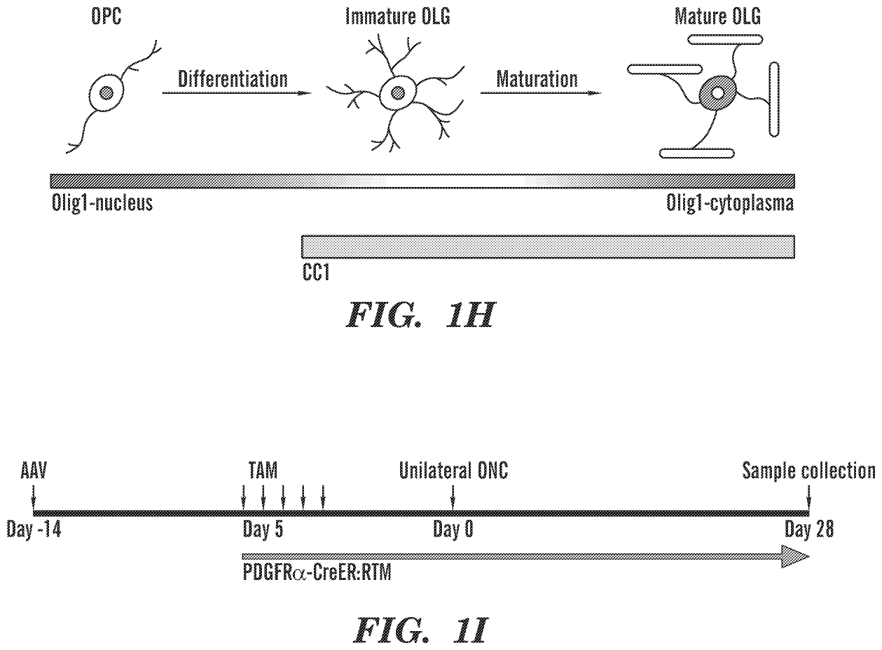

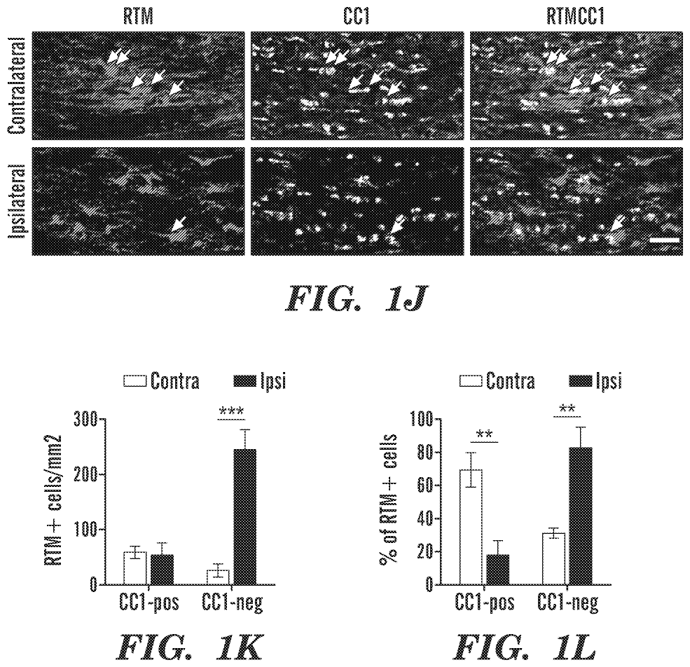

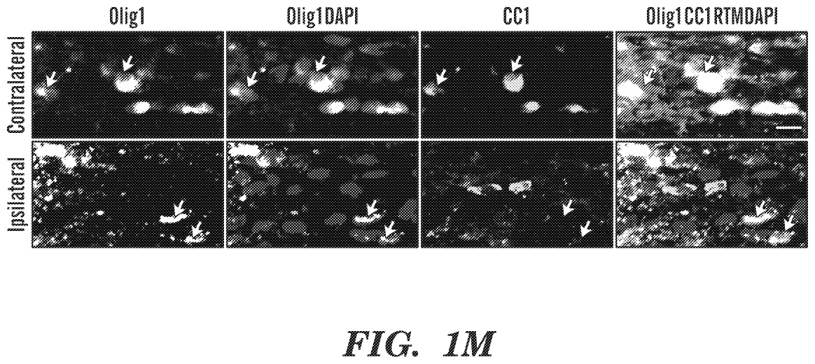

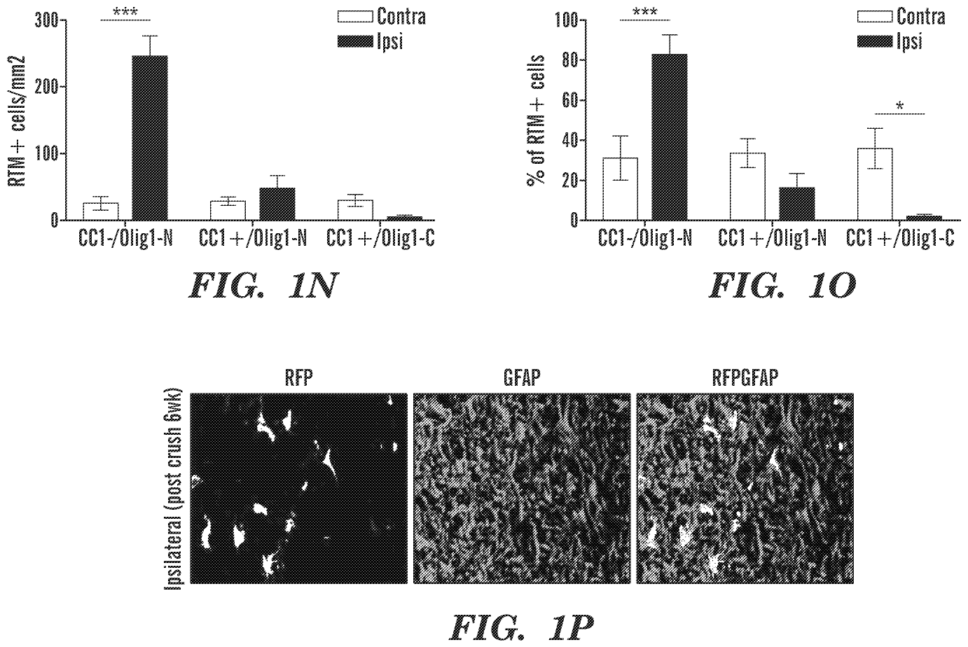

It is known that different manipulations could elevate the intrinsic regenerative ability of retinal ganglion cells (RGCs) and enabled robust axon regeneration after an optic tract injury, but none of these regenerated axons co-stained with antibodies against myelin associated glycoprotein (MAG). To further assess the myelination status of such regenerated axons induced by PTEN deletion, electron microscopic analysis of injured nerves was performed at 4 weeks after an optic nerve crush injury, in which all RGC axons are severed (Park et al., Science 322, 963-966 (2008)). As expected, many regenerated axons with the morphology indistinguishable from naïve axons could be seen in the optic nerves distal to the lesion (FIG. 1A). However, among several thousands of axons analyzed, only one regenerated axon had a thin layer of myelin (FIG. 1A). Thus, similar to the optic tract injury model, most regenerated axons do not exhibit spontaneous myelination in injured optic nerves.

As OPCs account for the myelination in the adult, how OPCs respond to injury was examined. First, proliferation of OPCs was assessed in injured (ipsilateral to crush) and control (contralateral) optic nerves of PDGFRα-H2B-GFP mice in which all OPCs express nuclear H2B-GFP. Because GFP in this reporter line is also expressed in <5% vascular and leptomeningeal cells, the optic nerve sections were co-stained with an oligodendrocyte lineage marker Olig2 and defined the GFP⁺ and Olig2⁺ double positive cells as OPCs (FIGS. 1B-E). As shown in FIGS. 1C and 1D, the total OPC numbers in the crushed nerves increased significantly at 1 and 2 weeks after injury, but returned to the basal levels at 4 weeks. In contrast, similar numbers of GFP⁺/Olig2⁺OPCs were seen in intact nerves at all time points (FIG. 1D). To further assess the dynamics of such injury-induced OPC proliferation, these mice were injected with BrdU at different time points after injury but terminated them at 3 hours after injury, with an expectation to label dividing OPCs at each time point (FIGS. 1F, 1G). The results indicated that injury-induced OPC proliferation was most significantly increased around 3-5 days after injury, became reduced at later time points, indicating a rapid yet reversible OPC proliferation triggered by optic nerve injury (FIG. 1G). Together, these results suggested that a crush injury triggers a rapid and reversible OPC proliferation in injured optic nerves.

### Example 2: Differentiation Failure of Proliferated OPCs in Injured Optic Nerves

It was next examined if these proliferated OPCs are able to undergo differentiation. To trace the progenies of OPCs, a different reporter mouse line was utilized, namely PDGFRα-CreER (Kang et al., Neuron 68, 668-681, 2010) crossed with Rosa26-STOP-Tomato mice, or PDGFRα-CreER/iRTM mice (Arenkiel et al., 2011). Upon tamoxifen administration, Cre expression is induced in PDGFRα⁺ OPCs, leading to RTM expression not only in these OPCs but also their differentiated progenies. To monitor their differentiation, two well-documented markers were used, namely CC1 and Oligo1. CC1 is a marker for all differentiated cells from OPCs. The translocation of Oligo1 from the nucleus to the cytoplasm has been implicated as a hallmark of late differentiation into myelinating oligodendrocytes. Therefore, with these immunohistochemistry assessments, these cells could be separated into three stages: un-differentiated OPCs (CC1⁻ and nuclear Oligo1), early differentiated OPCs (CC1+ and nuclear Oligo1) and mature oligodendrocytes (CC1⁺ and cytoplasmic Oligo1) (FIG. 111).

Immediately prior to optic nerve crush, tamoxifen was injected into PDGFRα-CreER/iRTM mice in order to label pre-existing OPCs (Young et al., Neuron 77, 873-885, 2013). Then, the fate of labeled OPCs was examined by immunohistochemistry with anti-CC1 and anti-Oligo1 at 4 weeks after injury (FIG. 1I). At 4 weeks after tamoxifen induction, although the total RTM⁺ number is lower in the intact contralateral optic nerves, 67% of RTM⁺ cells became CC1+ oligodendrocytes and about half of them exhibited cytoplasmic Oligo1⁺ (FIGS. 1J-L). This is consistent with the notion of continuous myelination in the adult. However, in the injured optic nerves of the same mice, only 17% RTM⁺ cells were CC1⁺ and most of these CC1⁺ cells had nuclear, but not cytoplasmic Oligo1 (FIGS. 1J-1O), suggesting undifferentiated OPCs. These results suggest that in injured optic nerves there are at least two blockades for OPCs to differentiate into mature oligodendrocytes, the early differentiation (becoming CC1⁺) and late maturation (with cytoplasmic Oligo1). In addition, although OPCs have been shown to have the potential of differentiating into astrocytes during development (Levison and Goldman, Neuron 10, 201-212, 1993), no RTM⁺ cells expressing astrocyte marker GFAP were observed (FIG. 1P). Thus, these data suggested that in contrast to intact optic nerves, proliferated OPCs exhibited differentiation blockades in injured nerves, reminiscent of what was observed in the lesions of progressive multiple sclerosis patients (Kuhlmann et al., Brain 132, 1118, 2008).

### Example 3: Injury-Induced GPR17 Upregulation Contributes to the Early Differentiation Failure of OPCs

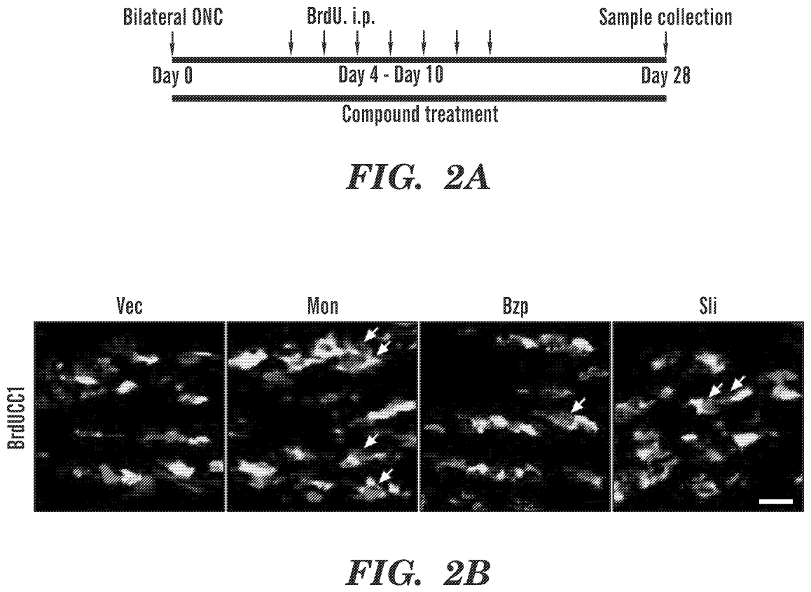

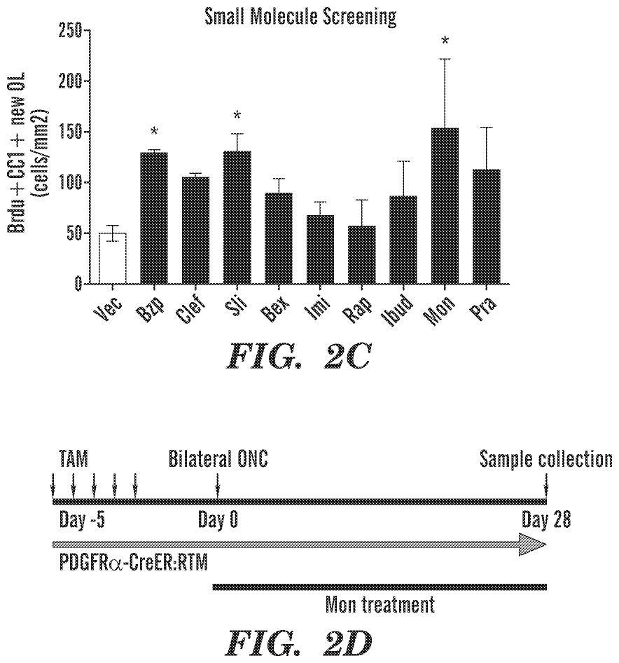

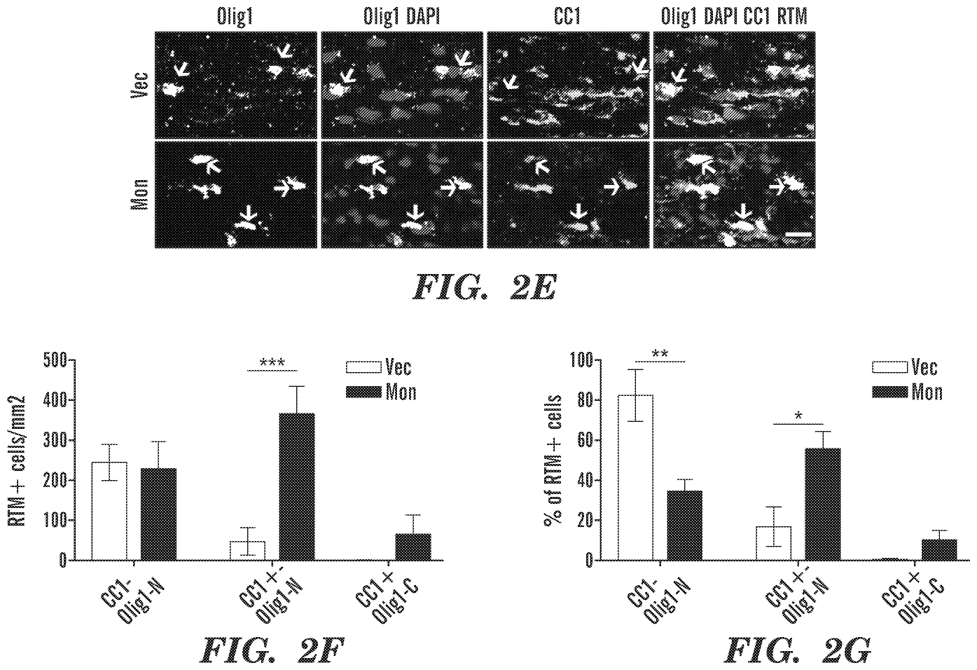

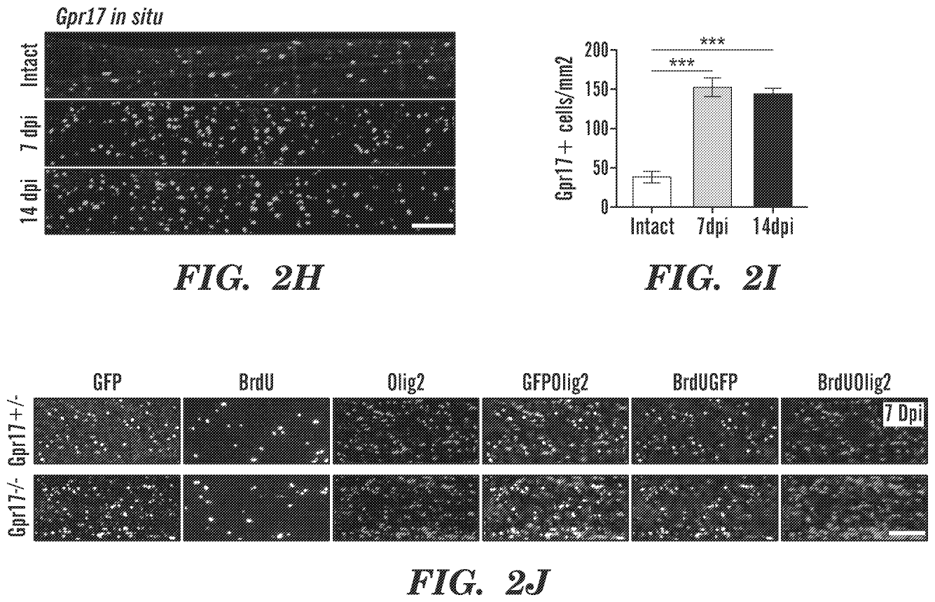

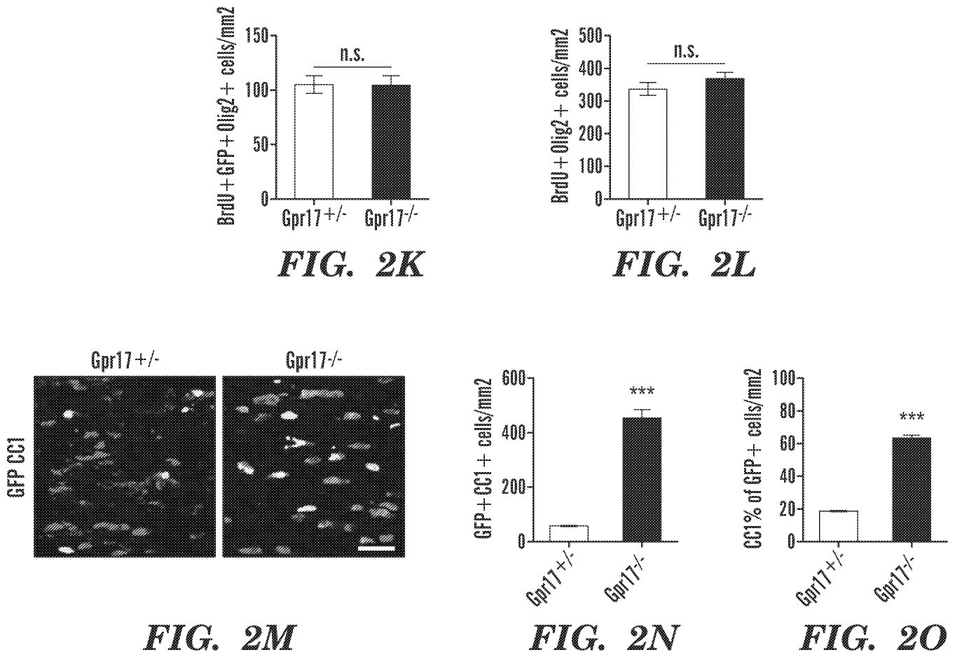

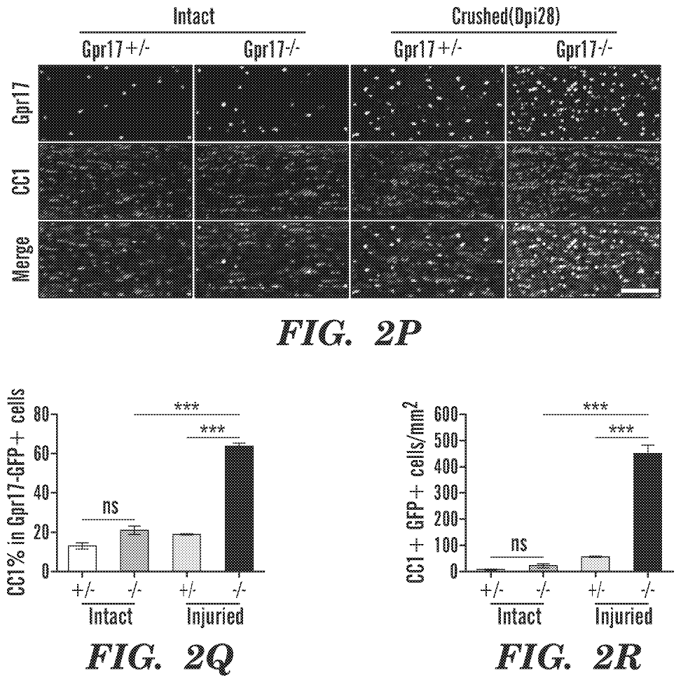

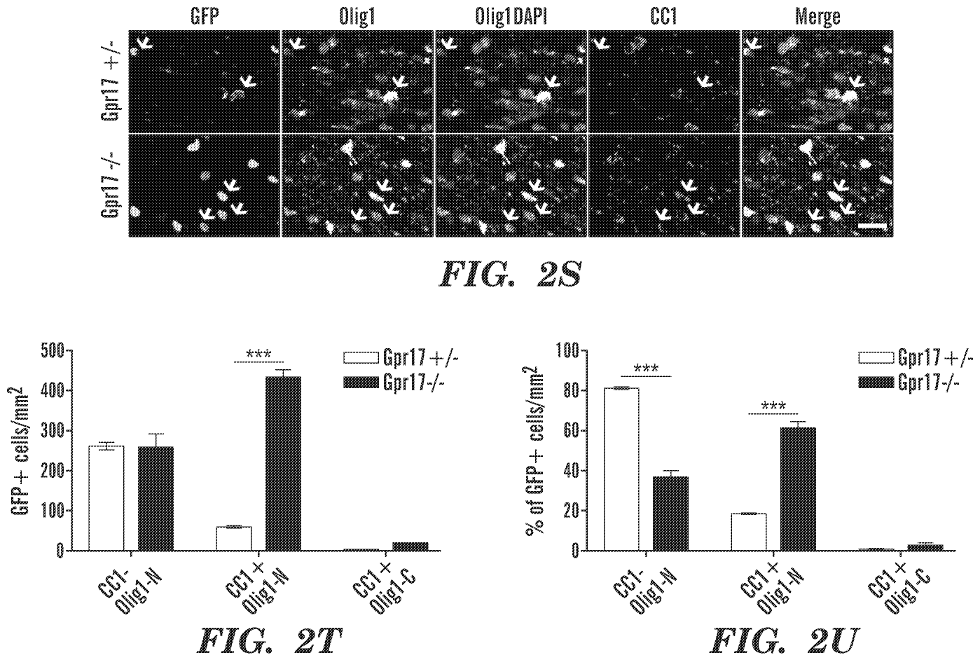

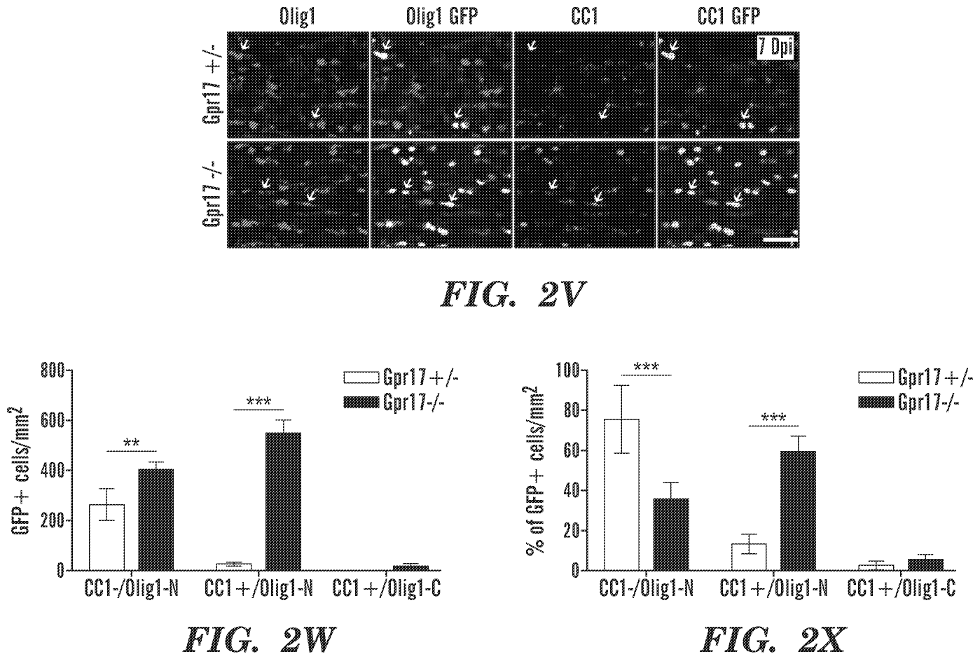

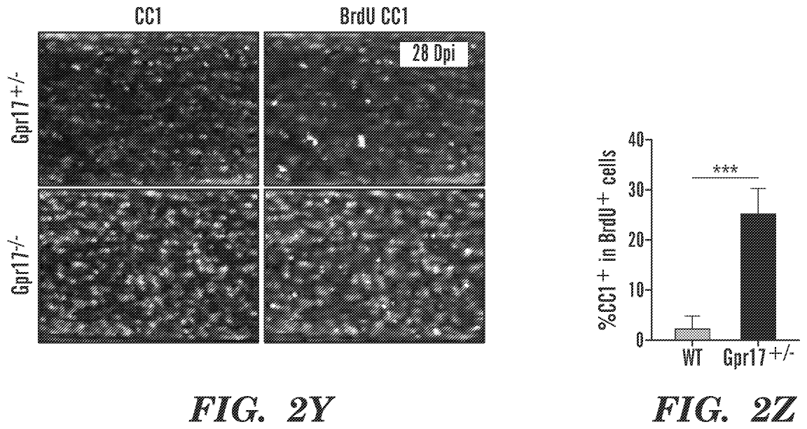

Based on in vitro and EAE models, previous studies identified a variety of compounds that could promote OPC proliferation and/or differentiation. However, it is unknown whether any of these agents could facilitate the myelination of regenerated axons. As a first step towards investigating such differentiation blockade of proliferated OPCs, a set of small molecule compounds were screened to identify ones that could increase differentiated OPCs in injured optic nerves (FIGS. 2A-2C). The candidate compounds include: benztropine mesylate (Bzp), a M1/M3 muscarinic receptor antagonist; clemastine (Clem), an antihistamine and anticholinergic agent, M1/M3 muscarinic receptor antagonist; Solifenacin (Sli), a M3 muscarinic receptor antagonist; Bexarotene (Bex), a retinoid X receptor agonist; imidazole (Imi), an anti-cholesterol synthesis compound; Ibudilast (Ibud), a clinically approved phosphodiesterase (PDE) inhibitor; and Montelukast (Mon) and Pranlukast (Pra), two different GPR17 antagonists. As rapamycin (Rap), a mTOR inhibitor, was shown to improve myelination in the mice of TSC1 knockout mice (Meikle et al., 2008), it was also included in our screening.

As all of these compounds could cross blood brain barrier, individual compounds were systemically administered for 4 weeks after optic nerve injury in wild type C57BL/6 mice. To trace the differentiation of proliferating OPCs, mice were injected daily with BrdU from days 4-10 after injury when OPCs exhibited high proliferation rate (FIG. 1G). In another 3 weeks (as differentiation may take 2-3 weeks), the pro-differentiation effect of each compound was evaluated by expression of oligodendrocyte marker CC1 on the BrdU⁺OPCs in injured optic nerves. As shown in FIGS. 2B and 2C, three compounds, including Montelukast, benztropine mesylate, and Solifenacin, significantly increased the number of BrdU⁺ and CC1⁺ double positive cells. Since Montelukast had the strongest effect, further studies focused on this compound and its putative target GPR17.

As a first step to verify this observation, Montelukast treatment was administered to PDGFRα-CreER/iRTM mice to selectively visualize OPCs and their progenies after optic nerve injury (FIG. 2D), similar to that in FIGS. 1H-M. After Montelukast treatment for 4 weeks, 64% of RFP⁺ cells became CC11, in contrast to 12% in vehicle-treated mice (FIGS. 2E-2G). Surprisingly, 77% of these CC1⁺RTM⁺ cells had nuclear, but not cytoplasmic, Oligo1 immunoreactivity (FIGS. 2E-2G). In addition, the total RTM⁺ cell numbers increased after Montelukast treatment (FIG. 2F). As the cell death is associated with failed differentiation of OPCs, such increased RTM⁺ cells may result from improved differentiation and reduced cell death. Together, these results suggested that Montelukast treatment could promote the early differentiation of OPCs, but these cells still fail to advance into mature oligodendrocytes.

Montelukast is a clinically approved treatment for treating asthma and seasonal allergies. Mechanistically, it acts as an antagonist of leukotriene receptors including the G protein-coupled receptor GPR17. In addition to Montelukast, another GPR17 antagonist Pranlukast (Pra) also increased OPC differentiation but did not reach statistical significance (FIG. 2C). This might be relevant to their different pharmacological properties, such as blood-brain barrier penetration property (Marschallinger et al., 2015). Nevertheless, these results strengthen the notion about the role of GPR17 in initiation of OPC differentiation. Intriguingly, previous studies showed that GPR17's expression is down-regulated in the adult CNS and the myelination appears normal in adult GPR17 knockout mice, although it was suggested as an intrinsic timer of OPC differentiation during development (Chen et al., 2009). Thus, in situ hybridization was used to assess the expression pattern of GPR17 in the optic nerves of different conditions. Consistent with previous reports (Chen et al., 2009), GPR17 expression was rarely detectable in intact optic nerves of adult mice (FIGS. 2H, 2I). However, optic nerve crush injury triggers significant upregulation of GPR17 in injured nerves, as detected at both 1 or 2 weeks after injury (FIGS. 2H, 2I).

As Montelukast can inhibit other leukotriene receptors in addition to GPR17, the effects of genetic deletion of GPR17 on OPC differentiation in injured optic nerves was assessed by utilizing a GPR17 knock-in mouse line (Chen et al., Nat. Neurosci. 12, 1398-1406, 2009). In this line, the GPR17 coding region is replaced by the sequence of histone 2b-fused GFP (H2b-GFP). Thus, these mice could be used for monitoring GPR17 expression (by GFP signal in both heterozygotes and homozygotes) and for loss-of-function studies (homozygotes). As expected, from 7 days after injury, GFP⁺ (GPR17⁺) cells significantly increased in both GPR17^{+/-} and GPR17^{-/-} mice (FIGS. 2J-2L). Most of these GFP⁺ were also co-stained with anti-Oligo2, consistent with their restricted expression in OPC lineage (FIG. 2J). By 30 days after injury, only 2.3% of GFP⁺ cells were CC1⁺ oligodendrocyte in the GPR17^{+/-} mice while 61% of GFP⁺ cells are CC1⁺ in GPR17^{-/-} mice (FIGS. 2M-2R). Consistent with Montelukast treatment, the majority of GPR17^{-/-} GFP⁺ cells showed Oligo1 immunoreactive signals in their nuclei, but not cytoplasm (FIGS. 2S-2U for dpi28 and FIGS. 2V-2X for dpi7). In addition, the number of GFP⁺ cells was significantly higher in GPR17^{-/-} mice compared to GPR17^{+/-} mice (FIGS. 2P-2R. By BrdU labeling, similar numbers of labeled cells were found in both groups (FIGS. 2J, 2L), suggesting that such different numbers of GFP⁺ cells are likely due to increased differentiation and thus reduced cell death upon GPR17 deletion. Consistently, at 4 weeks after injury, about ten-fold increase of BrdU⁺CC1⁺ cells were observed in GPR17^{-/-} mice versus GPR17^{+/-} mice (FIGS. 2Y and 2Z). Thus, similar to Montelukast treatment, GPR17 knockout facilitated the initial differentiation, but not late maturation of proliferated OPCs in injured optic nerves.

### Example 4: Differential Effects of Acutely or Sustained Activated Microglia on OPC Proliferation and Maturation

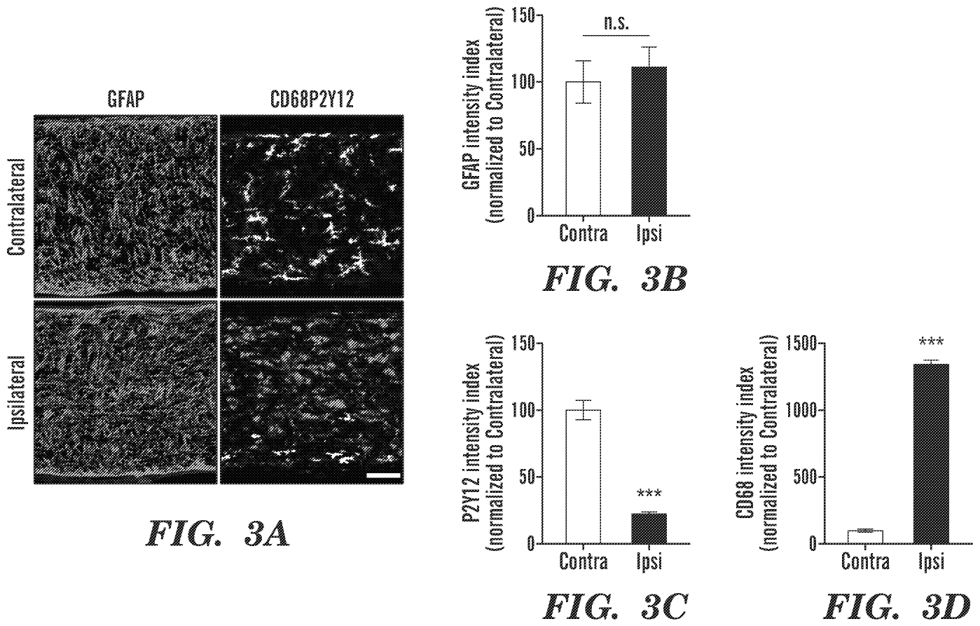

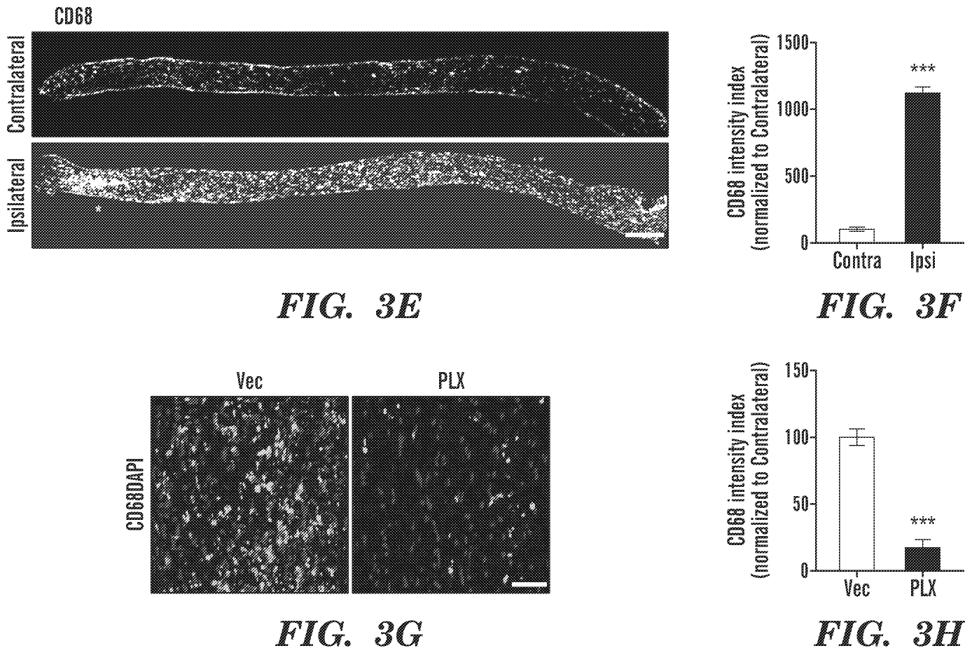

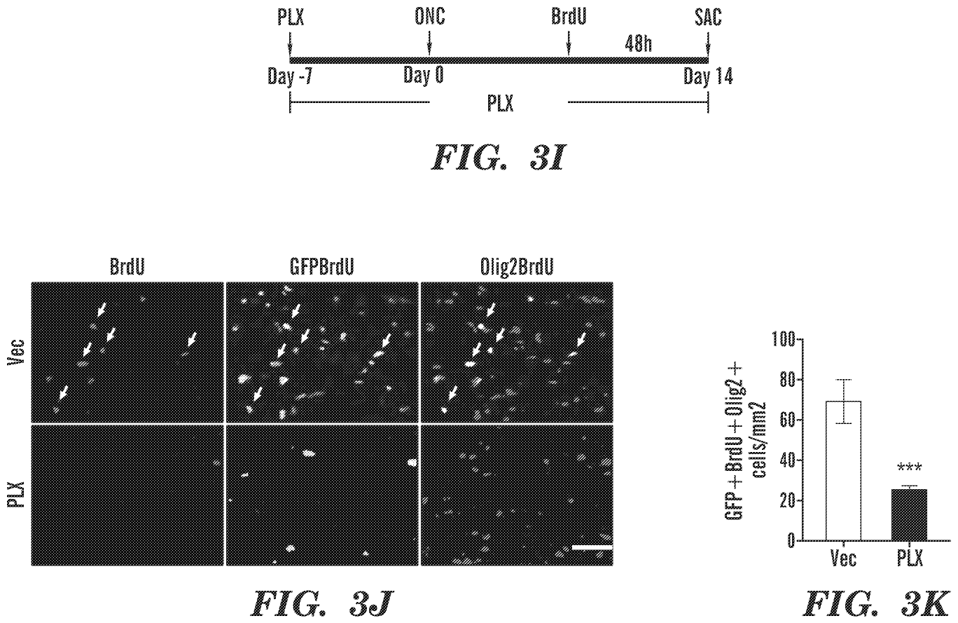

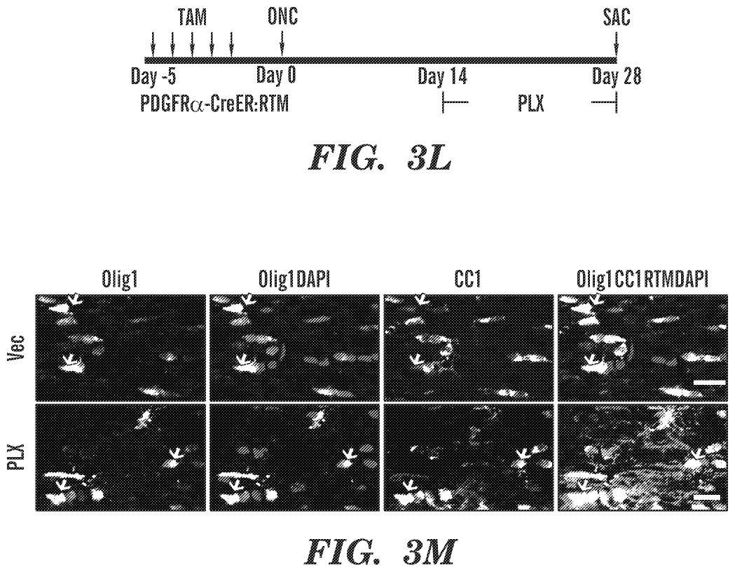

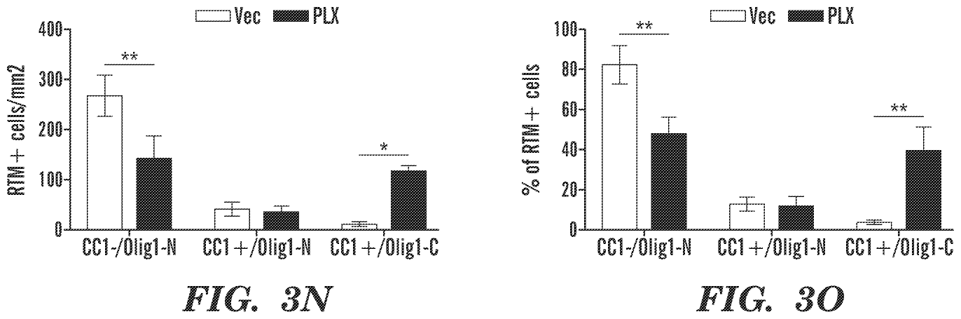

In light of the observed partial effects of GPR17 inhibition on OPC differentiation, additional blocker(s) of the late maturation step of differentiation of OPCs were sought. An important hint was different numbers of CC1⁺ cells with cytoplasmic Oligo1 in injured (ipsilateral) and their control uninjured (contralateral) nerves (FIGS. 1M-1O), suggesting a possible contribution of environmental factors to this late differentiation blockade. Consistent with well-characterized inflammation and chronic Wallerian degeneration in injured, but not uninjured optic nerves, microglia became rapidly and sustainably activated in injured optic nerves, as demonstrated by being positively stained with anti-CD68 antibodies and lacking immunoreactivity with anti-P2Y12, a marker for homeostatic microglia (FIGS. 3A-E). As inflammation has been suggested to regulate OPC proliferation and differentiation, the role of microglia in injured optic nerves on OPC proliferation and differentiation was further examined.

Taking advantage of the observation that systemic application of PLX3397, a colony stimulating factor 1 receptor (CSF1R) inhibitor, was able to specifically deplete microglia in vivo (FIGS. 3G, 3H), PDGFRα-H2B-GFP mice were pretreated with PLX3397 or its control for 7 days before optic nerve crush and examined for OPC proliferation at 14 days after injury by BrdU/GFP/Olig2 labeling (FIG. 3I). As shown in FIGS. 3J and 3K, PLX3397 treatment profoundly reduced the total numbers of OPCs, leaving few cells to be analyzed for their differentiation. Thus, it appears that microglia activation is required for injury-induced OPC proliferation.

As the majority of OPC proliferation occurs in the first week after injury (FIG. 1), it was hypothesized that a delayed PLX3397 treatment at 2-4 weeks after injury could bypass their inhibition on proliferation, permitting assessment of its effects on OPC differentiation. To do this, a separate experiment was performed in which PLX3397 was administered from 2 to 4 weeks after injury in PDGFRα-CreER:iRTM mice as used in FIGS. 1G-1M, 3L. As shown in FIGS. 3M-3O, PLX treatment increased CC1⁺cells. Importantly, among these CC1⁺RTM⁺ cells, 78% expressed cytoplasmic Oligo1, suggesting that delayed ablation of microglia promoted the maturation of early differentiated OPCs into myelinating oligodendrocytes.

### Example 5: Combinatorial Treatment of Montelukast and PLX3397 LED to Robust Myelination Regenerated Axons

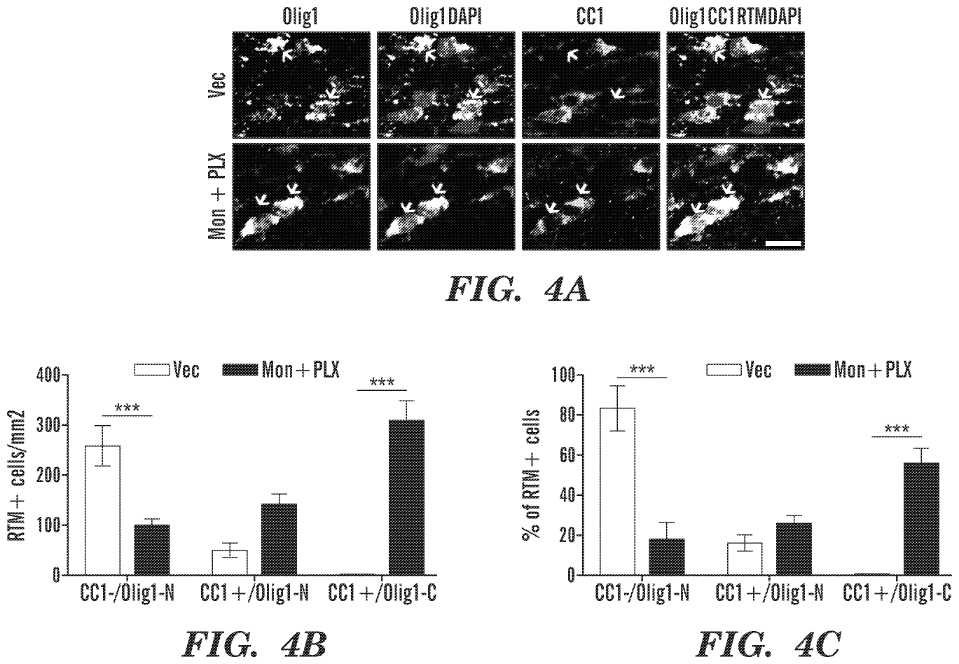

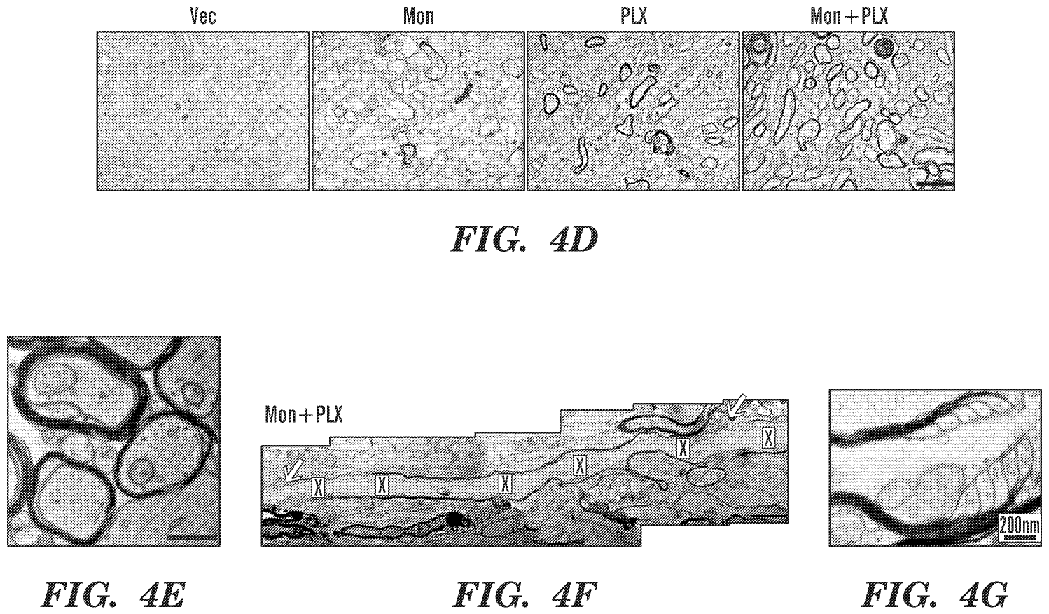

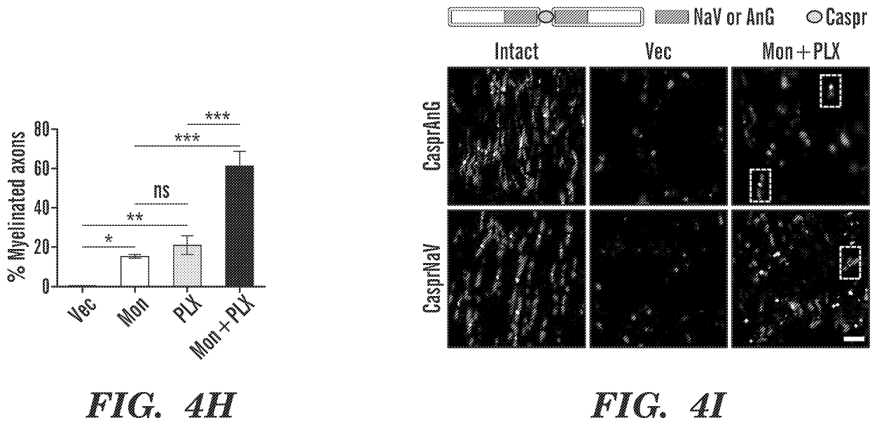

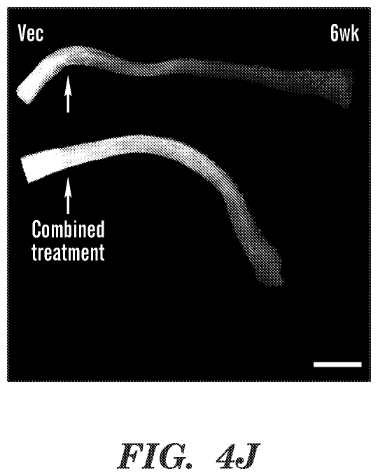

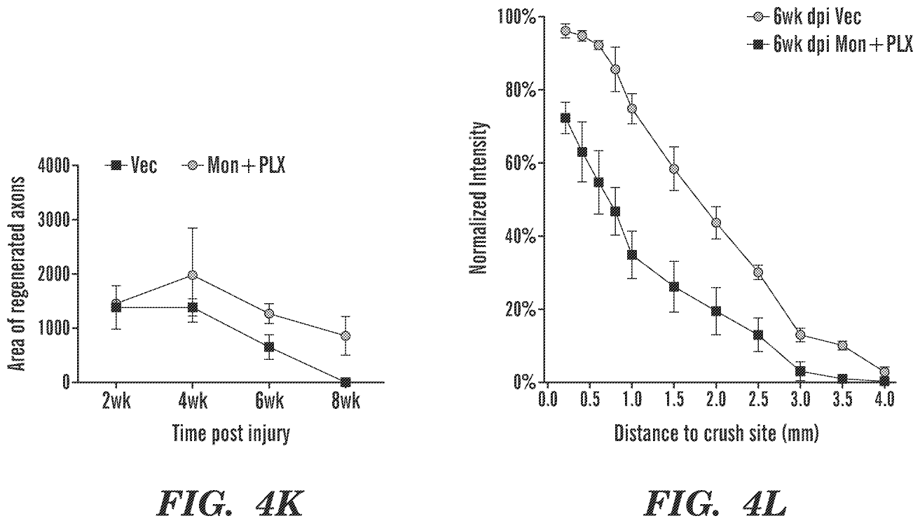

The observations regarding differential effects of GPR17 inhibition and delayed microglia ablation on OPC differentiation prompted examination of the effects of combined treatments on the myelination of regenerated axons. To do this, AAVs expressing osteopontin/IGF1/CNTF were injected into the vitreous bodies of PDGFRα-CreER:iRTM mice to activate the intrinsic regenerative ability of RGCs and optic nerve injury was performed in 2 weeks. These mice were then treated with Montelukast (for 4 weeks from dpi1 to dpi28) and/or PLX3397 (for two weeks during dpi 15-28). As shown in FIGS. 4A-4C, the combined treatment dramatically increased the numbers of CC1⁺RTM⁺ double positive cells and the majority of these CC1⁺ cells had cytoplasmic Oligo1, supporting the notion that this combinatorial treatment promoted both early and late differentiation of OPCs.

Some of mice in each group were subjected to electron microscopic analysis (FIGS. 4D-411) and additional immunohistochemistry (FIG. 4I). As shown in FIGS. 4D and 411, some (about 20%) regenerated axons became myelinated in the mice with the treatment of either Montelukast or PLX3397. However, the myelin structures after montelukast treatment were much thinner than those after PLX treatment, consistent with the results that montelukast or PLX3397 promotes the generation of early differentiated OPCs (with the ability of ensheathing axons and mature oligodendrocytes (with the ability of forming mature myelin), respectively. In contrast, the majority (60%) of regenerated axons were myelinated in the mice with the combined treatment (FIGS. 4D and 411). Many of these myelin structures are relatively thin and have large inner tongues, suggesting ongoing new myelination (FIG. 4E). However, the nodes of Ranvier could be clearly observed (FIGS. 4F and 4G). Consistently, nodes of Ranvier were observed and sometimes semi-nodes by immunohistochemistry with well-established markers, including Caspr, a component of the paranodal axoglial junction, Nav, and ankyrin G, two components of nodes of Ranvier (FIG. 4I). It is interesting to note that most of these regenerated axons have not crossed the optic chiasm, suggesting that such myelination occurs before these regenerated axons form functional synapses with their functional targets. Intriguingly, significantly more and longer regenerated axons with myelination-promoting treatments were observed (FIGS. 4J-4L), possibly relevant to the protective effects of myelination on axons. Together, these studies established a combined treatment that enables robust myelination of regenerated axons in injured optic nerves with sustained inflammation.

### Example 6: TNFα Expression is Upregulated by Optic Nerve Injury

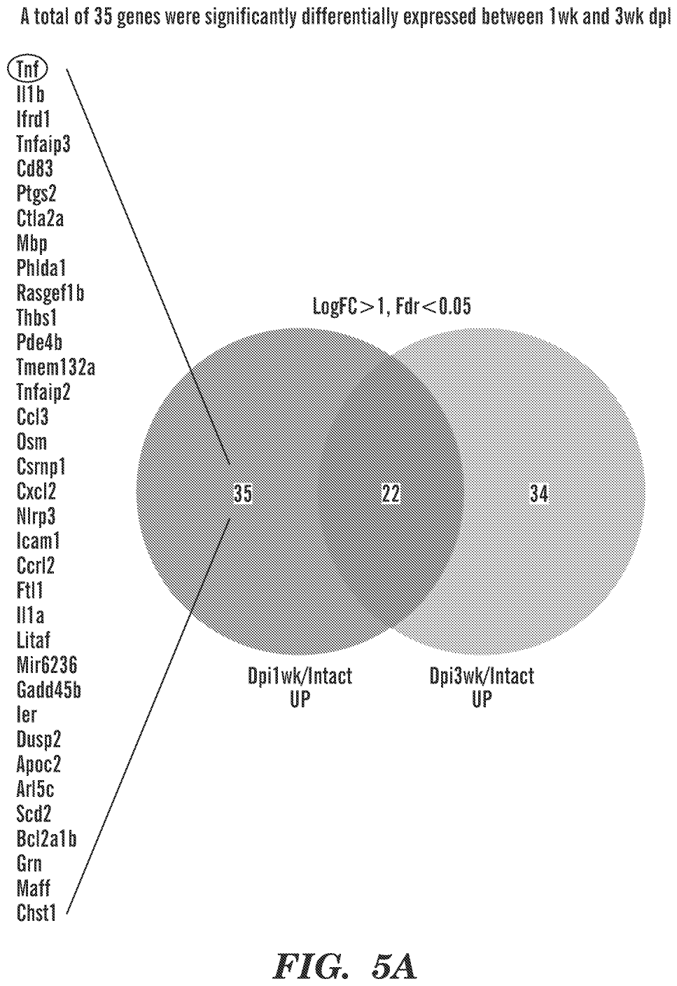

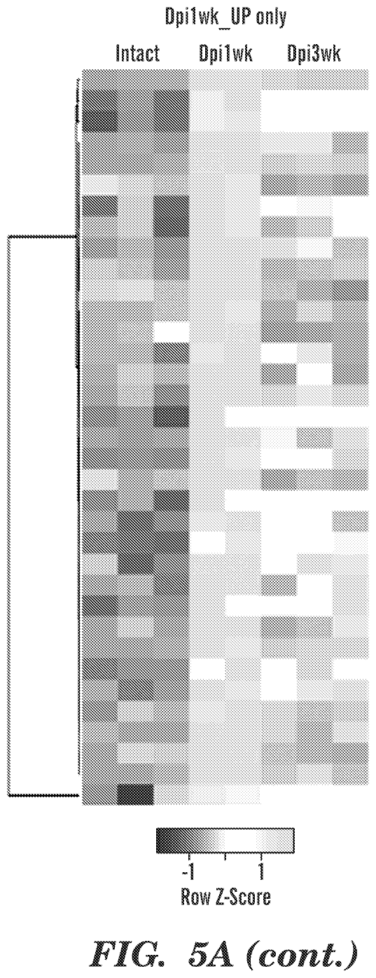

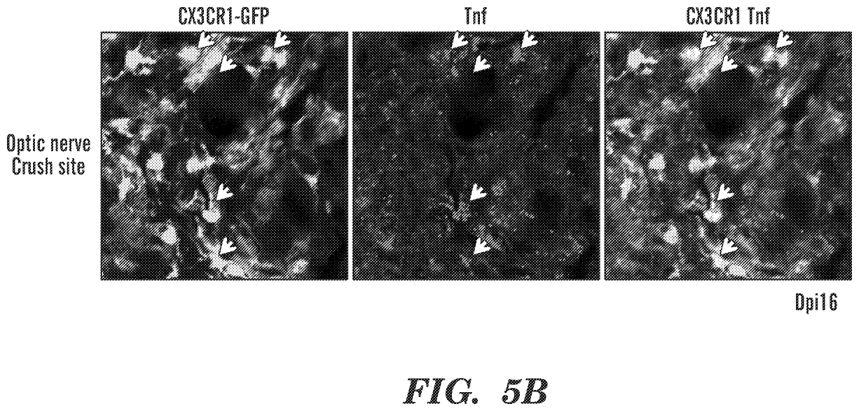

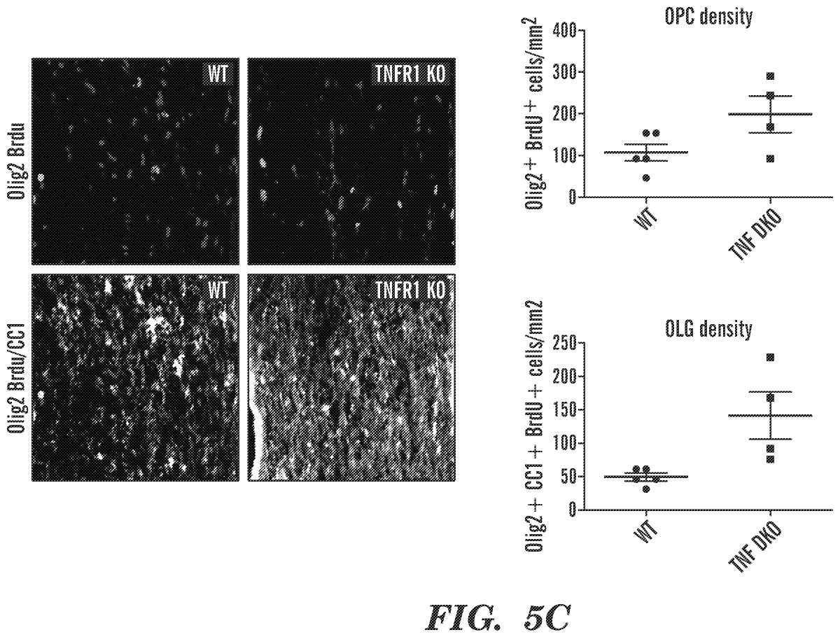

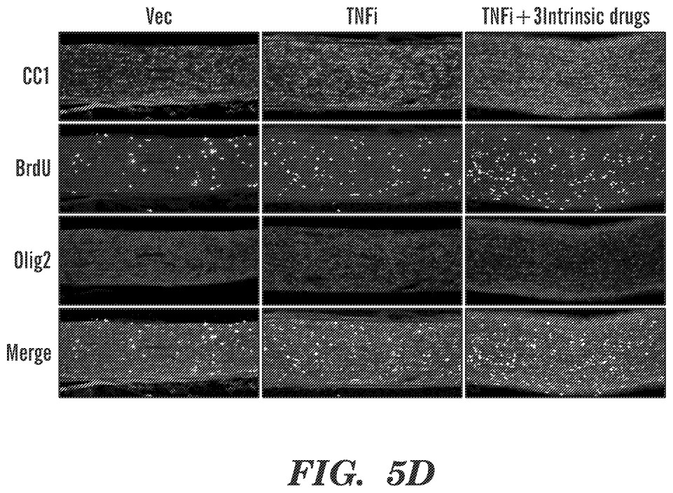

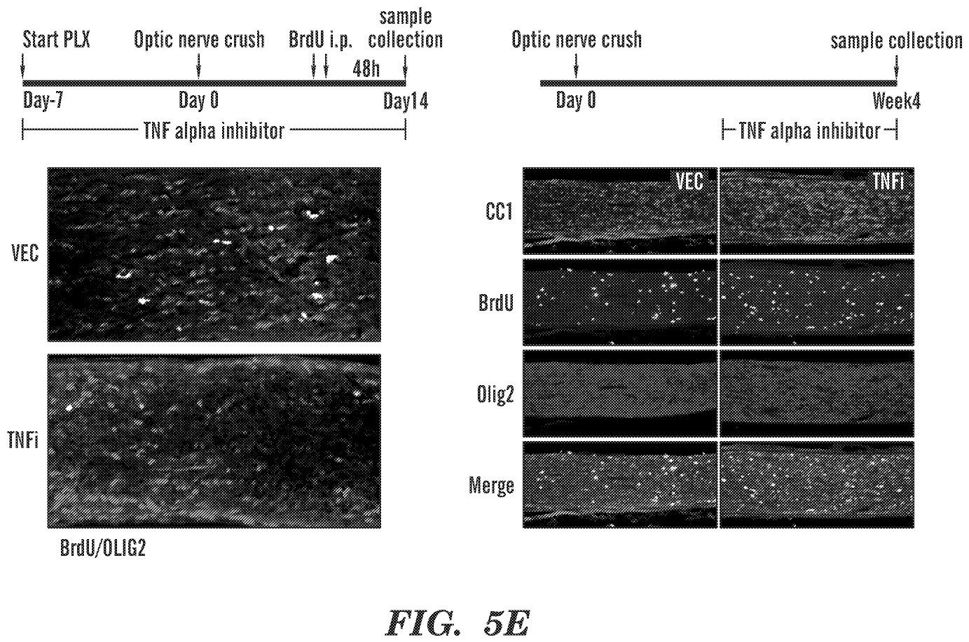

Sequencing data showed that TNF expression was upregulated following optic nerve injury at 1 week and 3 weeks post injury (FIGS. 5A, 5B). TNFR1 KO mice data showed robust increases of CC1⁺ oligodendrocytes at distal regions near crush site compared with heterozygous mice in the same litter (FIG. 5C). By using a BrdU pulse-chase protocol, significantly more BrdU⁺Olig2⁺ cells were observed in these mice. This indicated that there was increased survival and/or differentiation of cells derived from the OPC progenitors. This effect was not due to increased OPC proliferation since 3 hours of BrdU labelling showed fewer BrdU⁺Olig2⁺ cells (FIG. 5C).

Thalidomide, a TNF inhibitor, was administered from 2 weeks to 4 weeks post-injury and a robust increase of BrdU⁺/CC1+/Olig2⁺ cells was observed distal to the injury. By using PDGFRα-CreER/iRTM lineage reporter mice, it was found that OPCs differentiated into mature myelinating oligodendrocytes in response to this treatment (FIG. 5D). The morphology is very distinct from other treatments, such as GPR17 antagonist treatment (FIG. 5E).

In analyzing the underlying mechanisms of myelination failure of regenerated axons after optic nerve injury models, it was discovered that OPCs exhibited rapid proliferation but fail to differentiate into mature myelination oligodendrocytes. The mechanistic studies revealed two different differentiation blockades mediated by strikingly distinct mechanisms: injury-induced GPR17 preventing the early differentiation of OPCs into CC1⁺ cells, and injury-activated microglia blocking the maturation step towards myelinating oligodendrocytes (with cytoplasmic Oligo1). Individual manipulations increased the myelination to some extents, but combinatorial manipulations led to robust myelination of regenerated axons, highlighting the importance of dealing with both intrinsic and extrinsic mechanisms simultaneously. Together with recent advances in promoting axon regeneration in adult CNS, these results provide important insights in addressing another major roadblocks towards rebuilding functionally meaningful neuronal circuits. Intriguingly, the OPC dynamics observed in injured optic nerves/tracts are remarkably similar to what reported in the lesions of patients with progressive multiple sclerosis, both with proliferated OPCs failing to differentiate into mature oligodendrocytes. As activated microglia are dominant in injured optic nerves and multiple sclerosis lesions, the results reported here might be highly relevant for designing myelination-promoting interventions for progressive MS patients.

A number of molecules have been implicated as critical regulator of OPC differentiation. Surprisingly, montelukast appears to be the strongest to promote the initial stage of OPC differentiation. Although montelukast could target GPR17 and other cysteinyl-leukotriene receptors, similar results observed in GPR17 knockout and montelukast treatment studies point to GPR17 as a most relevant target. In this regard, it was shown that GPR17 is dramatically upregulated in injured optic nerves, most in early OPC lineage cells but in very few CC1⁺ cells, consistent with previous reports (Chen et al., 2009; Fumagalli et al., 2011). However, GPR17 inhibition promoted the majority of these cells (GFP⁺ in the transgenic mice) to be differentiated into CC1⁺ cells. Interestingly, the cell numbers from this lineage are also significantly increased after GPR17 inhibition. As this was not observed in knockout mice during development, this might be relevant to injury-associated factors. GPR17 is activated by cysteinyl-leukotrienes, thus, inflammation-elicited factors may also activate GPR17, preventing GPR17-expressing OPCs from differentiation and even proliferation. It is important to note that several other molecules may also play a role in this process, as two other M1/M3 muscarinic receptor antagonists, benztropine and solifenacin, also significantly increased OPC differentiation. In addition, another M1/M3 muscarinic receptor antagonist clemastine and the retinoid X receptor agonist Bexarotene also increased CC1⁺ cells, albeit the increase did not reach statistical differences.

The results presented herein also indicate a binary role of microglia in OPC dynamics: acutely activated microglia stimulating OPC proliferation but chronically activated microglia instead inhibit OPC differentiation, in particular the maturation step towards myelination oligodendrocytes. In fact, the link between microglia and myelination has been proposed. After It has been reported that inflammation is able to stimulate myelination by transplanted OPCs, and a number of studies pointed to microglia as an important regulator of myelination. More recently, it has been shown that chemotherapy such as Methotrexate results in persistent activation of microglia, which contributes to the impairment of OPC differentiation.

Together, the presently disclosed studies show that only co-manipulation of both intrinsic (GPR17) and extrinsic (microglia) factors is able to achieve robust myelination of regenerated axons. Future studies will examine whether such treatments enhance behavioral improvements in different injury models. Importantly, defective myelination is also associated with neurodegenerative diseases such as multiple sclerosis leukodystrophies, neurodegenerative Alzheimer's disease, and central nervous system injuries (e.g., traumatic brain injury, spinal cord injury) associated with myelination failure. As neuroinflammation might present in these conditions, it will be interesting to examine the activation states of microglia and test the effects of our manipulations on these conditions.

The results reported herein above were obtained using the following materials and methods.

### Mouse Strains

All experimental procedures were performed in compliance with animal protocols approved by the Institutional Animal Care and Use Committee at Boston Children's Hospital. Gpr17 transgenic mice were from Dr. Richard Lu (Chen et al., 2009). The other mouse strains were obtained from The Jackson Laboratory (Table 1). Experiments started when mice reached 6-8 week old. Both male and female mice were randomized and assigned to different treatment groups, prior to injury, and no other specific randomization was used for the animal studies. Quantifications were examined blindly.

### Antibodies

Primary antibodies used were: Rabbit anti-Oligo1(1:50, a gift from Dr. Charles D Stiles), Rabbit anti-Olig2 (1:300, Novus biologicals, NBP1-28667), Rat anti PDGFRa (CD140a) (1:100, BD Bioscience, 558774), Mouse anti-CC1(APC) (1:100, Millipore, OP80), Rat anti-BrdU(1:300, Abcam, ab6326), Mouse Anti-Nav1.6 (1:50, Antibodies incorporated, 75-026), Mouse anti-Ankyrin-G (AnkG) (1:50, Antibodies incorporated, 75-146), Rabbit anti-Caspr (1:1000, Abcam, ab34151), Rat anti-MBP(1:300, Abcam, ab7349), Mouse anti-MAG(1:100, Millipore, MAB1567), Rat anti-CD68(1:300, Bio-Rad, MCA1957), Rabbit anti-Ibal(1:500, WAKO Pure Chemicals, 019-19741), Rabbit anti-P2Y12 (1:500, AnaSpec, AS-55043A), Rat anti-GFAP (1:1000, ThermoFisher, 13-0300), Rabbit anti-RFP(1:500, Abcam, ab34771). Secondary antibodies (Invitrogen), raised in either goat against primary antibody's host species, highly cross adsorbed and conjugated to fluorophores of Alexa Fluor 488, Alexa Fluor 594, or Alex Fluor 647, and used at a 1:500 dilution.

### METHOD DETAILS

### Virus Production

All AAV viral vectors were made by Boston Children's Hospital Viral Core. AAV serotype 2 were used in the study as following: AAV2-Cre; AAV2-CNTF; AAV2-IGF1; AAV2-OPN. The titers of all viral preparations were at least 1.0×10¹³ GC/mL.

### Surgical Procedures

For all surgical procedures, mice were anaesthetized with ketamine and xylazine and received Buprenorphine as a postoperative analgesic.

### AAV Virus Injections

As previously described, intravitreal virus injection was performed two weeks before optic nerve crush injury to enable axon regeneration. Briefly, a pulled-glass micropipette was inserted near peripheral retina behind the ora serrata and deliberately angled to avoid damage to the lens. 2 μl of AAV2/2-CAG-Cre virus was injected for Pten f/f mice (Park et al., 2008). 2 μl of the combination of AAV2/2-CAG-CNTF, AAV2/2-CAG-IGF and AAV2/2-CAG-OPN (1:1:1 mix) was injected for other mouse strains (Bei et al., 2016).

### Optic Nerve Injury

As previously described, optic nerve was exposed intraorbitally and crushed with fine forceps (Dumont #5 FST) for 5 seconds, approximately 500 μm behind the optic disc. Afterwards, eye ointment was applied post-operatively to protect the cornea. Robust axon regeneration could be observed from 2 weeks post crush by Alexa-conjugated cholera toxin subunit B labeling.

### Compound Administration

For PDGFRα-CreER mice, Tamoxifen (100 mg/kg, p.o.) was administered for 5 days right before optic nerve crush. For OPC proliferation assays, BrdU (100 mg/kg, i.p.) was injected at 3 hours before sample collection. For drug screening assays, BrdU was injected daily from day 4 to day 10 post optic nerve crush. Each compound or the corresponding Vehicle was administered once daily for 4 weeks started from day 1 post optic nerve crush (Table 2). As previously described, the dose and route for tested compounds are: Bexarotene (100 mg/kg, p.o.), Benztropine mesylate (10 mg/kg, i.p.), Clemastin fumarate (10 mg/kg, p.o.), Ibudilast (10 mg/kg, i.p.), Imidazole (10 mg/kg, i.p.), Montelukast (25 mg/kg, p.o.), Pranlukast (0.5 mg/kg, i.p.), Rapamycin (6 mg/kg, i.p.), Solifenacin succinate (20 mg/kg, i.p.). Pexidartinib (PLX 3397) was mixed in food chow at 290 mg/kg by LabDiet laboratory animal nutrition.

### Perfusions and Tissue Processing

For immunostaining, animals were given an overdose of anesthesia and transcardiacally perfused with ice cold PBS followed by 4% paraformaldehyde (PFA, sigma). After perfusion, optic nerves were dissected out and postfixed in 4% PFA overnight at 4° C. Tissues were cryoprotected by sinking in 30% sucrose in PBS for 48 hours. Samples were frozen in Optimal Cutting Temperature compound (Tissue Tek) using a dry ice and then sectioned at 12 mm for optic nerves.

### Immunostaining and Imaging Analysis

Cryosections (12-μm thick) were permeabilized and blocked in blocking buffer (0.5% Triton X-100 and 5% normal goat serum in PBS) for 1 h at room temperature and overlaid with primary antibodies overnight at 4° C. (Table 1). For BrdU staining, cells or tissue sections were denatured with 2 N HCl for 30 minutes at 37° C. and then were neutralized with 0.1 M sodium borate buffer for 10 min before proceeding to normal blocking procedure. The following day, the corresponding Alexa Fluor 488-, 594- or 647-conjugated secondary antibodies were applied (all secondary antibodies were purchased from Invitrogen). All stained sections were mounted with solutions with DAPI-containing mounting solution and sealed with glass coverslips. All immunofluorescence-labeled images were acquired using Zeiss 700 or Zeiss 710 confocal microscope. For each biological sample, 3-5 sections of each optic nerve were imaged under 10× or 20×objectives for quantification. Positive cell numbers were then quantified manually using the Plugins/Analyze/Cell Counter function in ImageJ software. For fluorescent intensity analysis, the images were first converted to 8-bit depth in ImageJ software and then, the mean intensity value was calculated by the build-in function: Analyze/Measure.

### Tissue Clearing, Imaging and Quantification of Optic Nerve Regeneration

Mice injected with fluorophore tagged Cholera Toxin B (CTB) were perfused with 4% paraformaldehyde. Dissected optic nerves were then subjected to a modified procedure from previously published iDISCO tissue clearing method, which rendered the optic nerves transparent for direct fluorescent imaging (Renier et al., 2014). This procedure has been tested for better preservation of CTB florescence and the least change of optic nerve shape during tissue clearing. For dehydration, optic nerve samples were incubated in dark for 0.5 h of 80% tetrahydrofuran (THF, Sigma-Aldrich 360589-500ML)/H₂O and then switched to 100% THE for 1 h. Then, samples were incubated in Dichloromethane (DCM, Sigma-Aldrich 270997-1L) for 20 min (nerves should sink to the bottom). Samples were finally switched to dibenzyl ether (DBE, Sigma-Aldrich 33630-250ML) until complete transparency (at least 3 h, but overnight is recommended). Transparent nerves can be stored in DBE without obvious fluorescence decay of CTB for at least 1 year. For imaging, processed nerves can be mounted in DBE and imaged under Zeiss 710 confocal microscope. Z-stask scanning and maximum projection of Z-stack images were used in order to capture all regenerated axons. For image analysis, fluorescent intensity profile along the nerve was generated by the build-in function of ImageJ: Analyze/Plot Profile. To calculate the integral of fluorescent intensity across the entire length of the nerve, a Matlab algorithm was developed to quantify the “area under curve” from the plot profile data generated by ImageJ.

### Electron Microscopy and Morphometric Analysis

Mice were perfused with 4% paraformaldehyde with 2.5% glutaraldehyde in 0.1 M sodium cacodylate buffer, pH 7.2. Optic nerves were dissected and fixed in the same fixative solution overnight. Samples were then processed by the Harvard EM core based on the following procedure: nerves were rinsed in PBS, postfixed in 1% OsO₄ in PBS for 1 h, dehydrated in a graded ethanol series, infiltrated with propylene oxide, and embedded in Epon. Semithin sections were stained with toluidine blue, and ultrathin sections were stained with lead citrate. Ultrathin sections were taken under JEOL 1200EX-80 kV electron microscope. The number of myelinated axons per nerve was analyzed in ultrathin sections at magnification 3,000× to 20,000×.

### In Situ Hybridization

To assess the expression pattern of Gpr17, in situ hybridization was performed by hybridization chain reaction (HCR) (Choi et al., 2018) with a commercial kit from Molecular Instruments containing a DNA probe set, a DNA HCR amplifier, and different buffers. To prepare sections for in situ hybridization, anesthetized mice were perfused with DEPC-PBS followed by 4% paraformaldehyde (PFA). Dissected optic nerves were fixed in 4% PFA overnight, dehydrated in 30% sucrose/DEPC-PBS at 4° C., embedded in OCT and cryosectioned with 14 μm. Tissues were permeabilized in 5% SDS for 20 min at room temperature (RT) and pre-hybridized in hybridization buffer for 3 hour at 37° C. Then the slides were incubated in pre-warmed hybridization buffer including probes (2.5 nM for each) at 37° C. overnight. After hybridization, slices were washed for 1 hour at 37° C. with wash buffer followed by 2×SSC for 15 min at RT. The amplification step was performed with B3 HCR amplifiers overnight at RT.

### Quantification And Statistical Analysis

The normality and variance similarity were measured by STATA (version 12, College station, TX, USA) before we applied any parametric tests. Two-tailed student's t-test was used for the single comparison between two groups. The rest of the data were analyzed using one-way or two-way ANOVA depending on the appropriate design. Post hoc comparisons were carried out only when the primary measure showed statistical significance. P-value of multiple comparisons was adjusted by using Bonferroni's correction. Error bars in all figures represent mean S.E.M. The mice with different litters, body weights and sexes were randomized and assigned to different treatment groups, and no other specific randomization was used for the animal studies.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of increasing myelination of an axon, the method comprising contacting an oligodendrocyte progenitor cell (OPC) in the presence of an axon with one of the following:
an agent that inhibits GPR17 and/or an agent that ablates and/or inhibits an activated microglia
an agent that inhibits GPR17 and/or an agent that inhibits TNFα Receptor 2 or TNFα;
thereby increasing myelination of the axon.

2-3. (canceled)

4. The method of claim 1, wherein the method increases OPC number and/or differention.

5. The method of claim 1, wherein the agent that inhibits GPR17 is Montelukast or Pranlukast; the agent that ablates or inhibits an activated microglia is PLX3397; the agent that ablates or inhibits an activated microglia is PLX3397; and the agent that inhibits TNFα Receptor 2 or TNFα is thalidomide.

6-7. (canceled)

8. A method of increasing myelination of an axon, the method comprising contacting a oligodendrocyte progenitor cell (OPC) in the presence of an axon with an agent selected from the group consisting of benztropine mesylate, clemastine, Montelukast, Pranlukast, and thalidomide, thereby increasing myelination of the axon.

9. The method of claim 1, wherein the method increases OPC number and/or differentiation.

10-12. (canceled)

13. The method of claim 1, wherein the agents are administered concurrently or sequentially.

14-15. (canceled)

16. The method of claim 1, wherein the agents are administered prior to, concurrent with, or subsequent to injury.

17-18. (canceled)

19. The method of claim 1, wherein the agents are administered for at least between 14 and 28 days.

20-21. (canceled)

22. A method of increasing myelination of an axon in a subject, the method comprising administering to the subject an agent that inhibits GPR17 and/or an agent that ablates or inhibits an activated microglia, thereby increasing myelination of the axon.

23. The method of claim 1, wherein the method increases OPC number and/or differentiation in a subject.

24. The method of claim 22, wherein the agent that inhibits GPR17 is Montelukast or Pranlukast; the agent that ablates or inhibits an activated microglia is PLX3397.

25. (canceled)

26. A method of increasing myelination of an axon in a subject in need thereof, the method comprising administering to the subject an agent selected from the group consisting of benztropine mesylate, clemastine, Montelukast, Pranlukast, and thalidomide, thereby increasing myelination of the axon.

27. The method of claim 26, wherein the method increases OPC number and/or differentiation in a subject in need thereof.

28. A method of treating a subject having a disease or injury associated with myelination failure, the method comprising administering to the subject an agent that inhibits GPR17 and/or an agent that ablates or inhibits an activated microglia.

29. (canceled)

30. The method of claim 27, wherein the subject has multiple sclerosis (MS), a leukodystrophy, neurodegenerative Alzheimer's disease, traumatic brain injury, spinal cord injury, or optic nerve injury.

31. The method of claim 30, wherein the leukodystrophy is Adrenoleukodystrophy (ALD), Aicardi-Goutieres Syndrome, Alexander Disease, Canavan Disease, Cerebrotendinous Xanthomatosis (CTX), Globoid Cell Leukodystrophy (Krabbe Disease), Metachromatic Leukodystrophy (MLD), Pelizaeus Merzbacher Disease (X-linked spastic paraplegia), and Childhood Ataxia with Central Nervous System Hypomyelination (CACH).

32. The method of claim 27, wherein the agents are administered concurrently or sequentially.

33-37. (canceled)

38. The method of claim 30, wherein the traumatic brain injury is a concussion.

39-41. (canceled)

42. A composition comprising a GPR17 antagonist and a microglia inhibitor or ablation agent or TNFα inhibitor.

43. The composition of claim 42, wherein the GPR17 antagonist is montelukast and the microglia inhibitor or ablation agent is PLX3397.

44. (canceled)

45. A method of identifying a compound that elicits differentiation of an oligodendrocyte or an oligodendrocyte precursor cell, the method comprising:
injuring the optic nerve of a mouse;
contacting the optic nerve with an agent that regenerates an axon;
administering a candidate compound to the mouse to elicit differentiation of an oligodendrocyte precursor cell;
administering a known microglia inhibitor or ablation agent; and
determining the differentiation status of oligodendrocytes or oligodendrocyte precursor cells, wherein an increase in CC1+ oligodendrocytes relative to an untreated control indicates that the candidate compound elicited differentiation of the oligodendrocyte precursor cell or wherein an increase in CC1+ oligodendrocytes having cytoplasmic Oligo1 relative to an untreated control indicates that the suspected microglia inhibitor or ablation agent effectively inhibited or ablated microglia cells.

46. (canceled)
